SEQUENCE LISTING (9 sequences)

SEQ ID NO: 1 - protein, 120 aa, synthetic construct [sequence omitted]

SEQ ID NO: 2 - protein, 106 aa, synthetic construct
DIQMTQSPASLSASVGDRVTITCRASENIFSYLAWYQQKPGKSPKLLVYNTRTLAEGVPSFSGSGSGTDFSLTISSLQPEDFATYYCQHHYGTPFTFGSGTKLEIK

SEQ ID NO: 3 - protein, 8 aa, synthetic construct
GFVFSSYD

SEQ ID NO: 4 - protein, 8 aa, synthetic construct
ISSGGGIT

SEQ ID NO: 5 - protein, 13 aa, synthetic construct
AAHYFGSSGPFAY

SEQ ID NO: 6 - protein, 6 aa, synthetic construct
ENIFSY

SEQ ID NO: 7 - protein, 9 aa, synthetic construct
QHHYGTPFT

SEQ ID NO: 8 - protein, 449 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0151502 A1
(43) Pub. Date: Jun. 4, 2026

(54) USE OF ANTI-CEACAM5 IMMUNOCONJUGATES FOR TREATING NEUROENDOCRINE CANCERS EXPRESSING CEACAM5

(71) Applicant: SANOFI, Paris (FR)

(72) Inventors: Anne-Laure Bauchet; Mustapha Chadjaa; Anne-Marie Lefebvre; Robert Mashal, Cambridge, MA (US); Jean-Yves SCOAZEC, Villejuif (FR)

(21) Appl. No.: 19/161,167

(22) PCT Filed: Feb. 29, 2024
(86) PCT No.: PCT/EP2024/055281

§ 371 Date: Aug. 29, 2025

(30) Foreign Application Priority Data
Mar. 1, 2023 (EP) 23305267.9

(51) Int. Cl. A61K 47/68; A61P 35/00; C07K 16/28

(52) U.S. Cl. CPC A61K 47/6853; A61K 47/68033; A61P 35/00; C07K 16/2803

(57) ABSTRACT

The present disclosure provides methods of treating neuroendocrine cancers using immunoconjugates comprising an antibody that specifically binds hCEACAM5, in particular hCEACAM5.

### FIELD

The present disclosure relates to the field of therapeutic treatment of cancers, such as neuroendocrine cancers, which express CEACAM5, in particular hCEACAM5. Certain aspects of the disclosure relate to the use of CEACAM5 antagonists, such as anti-CEACAM5 antibodies and immunoconjugates, to treat neuroendocrine cancers.

### BACKGROUND

The mechanism of action of antibody drug conjugates (ADCs) begins with its binding to a specific antigen, sufficiently expressed on the tumor cells in order to achieve a selective and efficient internalization of the drug. Selectively targeting potent cytotoxic to tumor cells using ADCs has now been shown to be an effective strategy for the treatment of cancer, as demonstrated by the recent approvals of brentuximab vedotin for the treatment of Hodgkin lymphoma and trastuzumab emtansine (T-DM1) for the treatment of relapsed metastatic HER2+ breast cancer. Many other malignant diseases with unmet medical needs, such as solid tumor cancers, could benefit from such therapeutic options.

Neuroendocrine carcinomas are highly malignant diseases, defined as neoplasms made of poorly differentiated cells expressing neuroendocrine markers (such as chromogranins, synaptophysin, INSM1, CD56 . . . ) and showing high proliferative capacities (testified by high mitotic and Ki-67 indexes). Neuroendocrine neoplasms are classified according to a WHO classification based on differentiation and proliferative grading.

Their management is currently based on a standard first line chemotherapy, combining etoposide and cisplatin; after an initial response, all patients will rapidly recur: second line treatments are poorly efficient and the mean survival is about 12-15 months.

It has been shown in the art that certain neuroendocrine cancers, such as prostate cancer and medullary thyroid cancer, express particular tumor markers. (Lee et al., PNAS, Mar. 28, 2018, 115 (19) E4473-E4482 and Turkdogan et al., Journal of Otolaryngology—Head and Nech Surgery, 2018, 47:55). Lee et al. suggests that the cell-surface proteins FXYD3 and CEACAM5 in particular may serve as targets for immune-based therapy against advanced prostate cancer.

Carcino-embryonic antigen (CEA) is a glycoprotein involved in cell adhesion. CEA is a protein normally expressed by fetal gut during the first six months of gestation, and found in cancers of the pancreas, liver and colon. The CEA family, which consists of 18 genes, is sub-divided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12).

In humans, the CEACAM sub-group consists of 7 members: CEACAM1, CEACAM3, CEACAM4, CEACAM5, CEACAM6, CEACAM7, CEACAM8. Numerous studies have shown that CEACAM5 is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach and squamous epithelial cells in esophagus and cervix (Hammarström et al, 2002, in “Tumor markers, Physiology, Pathobiology, Technology and Clinical Applications” Eds. Diamandis E. P. et al., AACC Press, Washington pp 375). Thus, CEACAM5 may constitute a therapeutic target suitable for tumor specific targeting approaches, such as antibody-drug conjugates (ADCs).

Numerous anti-CEA antibodies were generated in view of CEA-targeted diagnostic or therapeutic purposes. Specificity towards related antigens has always been mentioned as a concern in this field. WO 2014/079886 has disclosed an antibody binding to the A3-B3 domain of human and Macaca fascicularis CEACAM5 proteins and which does not significantly cross-react with human CEACAM1, human CEACAM6, human CEACAM7, human CEACAM8, Macaca fascicularis CEACAM1, Macaca fascicularis CEACAM6, and Macaca fascicularis CEACAM8. This antibody has been conjugated to a maytansinoid, thereby providing an antibody-drug conjugate (ADC) (or immunoconjugate) having a significant cytotoxic activity on MKN45 human gastric cancer cells, with IC₅₀ values≤1 nM.

Use of anti-CEACAM-SN38 antibody-drug conjugates have been described in the art and suggested as therapeutic agents in the treatment of neuroendocrine prostate cancer (DeLucia et al., Clin Cancer Res, 2021, February 01, 27(3): 759-774). These conjugates have specific structures and mechanisms of action. SN-38 is an irinotecan analog derived from camptothecin and being a DNA topoisomerase type I inhibitor.

The development of new therapeutic and more effective strategies to treat CEACAM5 expressing neuroendocrine cancers is an ongoing need.

### SUMMARY

This disclosure provides, inter alia, methods for treating neuroendocrine cancer in a subject in need thereof comprising administering an effective amount of an antibody or immunoconjugate (comprising the antibody) that specifically binds CEACAM5.

In some embodiments, the disclosure relates to an immunoconjugate comprising an antibody or an antigen-binding fragment thereof, for use in treating a cancer selected from neuroendocrine cancers expressing hCEACAM5, in a subject in need thereof, wherein said antibody or antigen-binding fragment thereof specifically binds hCEACAM5, and wherein said antibody or antigen-binding fragment thereof is conjugated or linked to at least one growth inhibitory agent, wherein said growth inhibitory agent is not a topoisomerase I inhibitor.

In some embodiments, the said antibody or antigen-binding fragment thereof comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

In some embodiments, the growth inhibitory agent is selected in the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, topoisomerase II inhibitors, DNA alkylating agents, anti-tubulin agents, and CC-1065 or CC-1065 analogs.

In some embodiments, the disclosure relates to an immunoconjugate comprising an antibody or said antigen-binding fragment thereof, for use in treating a cancer selected from neuroendocrine cancers expressing hCEACAM5, in a subject in need thereof, wherein said antibody or antigen-binding fragment thereof specifically binds hCEACAM5, wherein said antibody or antigen-binding fragment thereof comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT)
wherein said neuroendocrine cancer is not a neuroendocrine small cell lung carcinoma (SCLC).

In some embodiments, a neuroendocrine cancer is selected from the group consisting of neuroendocrine cancers of the oesophagus, the stomach, the small intestine and the large intestine, the anal region, the pancreas, the bladder, the female reproductive organs, the male reproductive organs, the thyroid and the head and neck.

According to another aspect, the present disclosure relates to an antibody, or an antigen-binding fragment thereof, or an immunoconjugate comprising said antibody or said antigen-binding fragment, for use in treating a cancer selected from neuroendocrine cancers expressing hCEACAM5 in a subject in need thereof, wherein the antibody specifically binds hCEACAM5 and wherein the antibody, or the antigen-binding fragment thereof, comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

In a particular embodiment, the cancer is selected from neuroendocrine tumors (NET), neuroendocrine carcinomas (NEC), Mixed Neuroendocrine-Non-Neuroendocrine Neoplasma (MiNEN), Pheochromocytoma and Medullary thyroid carcinoma (MTC).

In particular, the cancer is selected from neuroendocrine cancers of:
the gastrointestinal and pancreatobiliary tract, such as neuroendocrine cancers of the digestive tract in particular the oesophagus, the stomach, the pancreas, the liver, the small intestine and large intestine or the anal region, or such as neuroendocrine cancers of the urinary tract, in particular the bladder;
the upper aerodigestive tract and salivary glands, such as neuroendocrine cancers of the lung, in particular a small cell lung cancer, and such as neuroendocrine cancers of the thymus;
the thyroid, such as medullary thyroid carcinoma (MTC);
the adrenal gland, such as pheochromocytoma;
the skin, such as Merkel cell carcinoma (MCC);
the female reproductive organs, such as of the endometrium, the uterine cervix or the ovary;
the male reproductive organs, such as of the prostate or the testis, in particular the testis, and
the head and neck.

In particular, the cancer is selected from neuroendocrine cancers of the oesophagus, the stomach, the small intestine and the large intestine, the anal region, the pancreas, the bladder, the lung, the female reproductive organs, the male reproductive organs, and the head and neck. More particularly, the cancer is a neuroendocrine cancer of the lung, in particular a neuroendocrine small cell lung cancer, or a neuroendocrine cancer of the male reproductive organs, in particular a neuroendocrine prostate cancer.

In some embodiments, the cancer is a neuroendocrine small cell lung cancer. In another embodiment, the cancer is neuroendocrine prostate cancer.

In some embodiments, the cancer is not a neuroendocrine small cell lung cancer. In another embodiment, the cancer is not neuroendocrine prostate cancer.

In a particular embodiment, the subject may be a moderate or high carcinoembryonic antigen-related cell adhesion molecule expresser. In particular, the subject is a moderate CEACAM5 expresser, that is a subject who has a CEACAM5 expression consisting of 2+ or 3+ intensity, measured by hCEACAM5 immunohistochemistry, in at least 1% of the tumor cell population or consisting of 1+ intensity in at least 50% of the tumor cell population. In particular, the subject may be a high CEACAM5 expresser, that is a subject who has a CEACAM5 expression consisting of 2+ or 3+ intensity, measured by hCEACAM5 immunohistochemistry, in over 50% of the tumor cell population.

In a particular embodiment, the VH domain comprises SEQ ID NO: 1:

EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSS.

The antibody or immunoconjugate may comprise a heavy chain comprising SEQ ID NO: 8:

EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

In a particular embodiment, the VL domain comprises SEQ ID NO: 2:

DIQMTQSPASLSASVGDRVTITCRASENIFSYLAWYQQKPGKSPKLLV
YNTRTLAEGVPSRFSGSGSGTDFSLTISSLQPEDFATYYCQHHYGTPF
TFGSGTKLEIK.

The antibody or immunoconjugate may comprise a light chain comprising SEQ ID NO: 9:

DIQMTQSPASLSASVGDRVTITCRASENIFSYLAWYQQKPGKSPKLLV
YNTRTLAEGVPSRFSGSGSGTDFSLTISSLQPEDFATYYCQHHYGTPF
TFGSGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC.

In a particular embodiment, the antibody of the immunoconjugate comprising the antibody is conjugated or linked to at least one growth inhibitory agent. In particular, said growth inhibitory agent may be a cytotoxic agent. In particular, said growth inhibitory agent may be selected from the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, topoisomerase inhibitors, DNA alkylating agents, anti-tubulin agents, and CC-1065 or CC-1065 analogs. In some embodiments, the topoisomerase inhibitors are not topoisomerase I inhibitors. In some embodiments, the topoisomerase inhibitors are topoisomerase II inhibitors.

In a particular embodiment, said growth inhibitory agent is (N²′-deacetyl-N²′-(3-mercapto-1-oxopropyl)-maytansine) (DM1) or N²′-deacetyl-N²′-(4-methyl-4-mercapto-1-oxopentyl)-maytansine (DM4).

In a particular embodiment, the antibody is covalently attached via a cleavable or non-cleavable linker to the at least one growth inhibitory agent.

In a particular embodiment, the immunoconjugate comprises a hCEACAM5-antibody, which comprises a heavy chain consisting of SEQ ID NO: 8 and a light chain consisting of SEQ ID NO: 9, and which is covalently linked to N²′-deacetyl-N²′(4-methyl-4-mercapto-1-oxopentyl)-maytansine (DM4) via N-succinimidyl pyridyldithiobutyrate (SPDB).

In a particular embodiment, the immunoconjugate is tusamitamab ravtansine.

In a particular embodiment, wherein the antibody or immunoconjugate is administered at a dose level of 5, 10, 20, 30, 40, 60, 80, 100, 120, 150, 180, or 210 mg/m2 based on the body surface area of the subject. In a particular embodiment, the antibody or immunoconjugate is administered every 2 weeks, or every 3 weeks. In a particular embodiment, the antibody or immunoconjugate is administered at a dose level of 100 mg/m2 based on the body surface area of the subject every 2 weeks. In a particular embodiment, the antibody or immunoconjugate is administered at a dose of from about 100 mg/m² to about 200 mg/m² as a loading dose, in particular of about 135 mg/m², about 150 mg/m² or about 170 mg/m², as a loading dose.

The text further describes a method for treating a cancer selected from neuroendocrine cancers expressing CEACAM5 in a subject in need thereof, the method comprising administering an antibody, or an antigen-binding fragment thereof, or an immunoconjugate comprising the antibody or said antigen-binding fragment, wherein the antibody specifically binds hCEACAM5 and wherein the antibody comprises a VH domain and a VL domain, wherein the VH domain comprises the three complementarity determining regions (CDRs) HCDR1, HCDR2 and HCDR3 and wherein the VL domain comprises the three CDRs: LCDR1, LCDR2 and LCDR3,
wherein the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY);
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

In particular, the cancer may be selected from neuroendocrine tumors (NET), neuroendocrine carcinomas (NEC), Mixed Neuroendocrine-Non-Neuroendocrine Neoplasma (MiNEN), Medullary thyroid carcinoma (MTC) and, Pheochromocytoma. In particular, the cancer may be selected from neuroendocrine cancers of:
the gastrointestinal and pancreatobiliary tract, such as neuroendocrine cancers of the digestive tract in particular the oesophagus, the stomach, the pancreas, the liver, the small intestine and large intestine, or the anal region, or such as neuroendocrine cancers of the urinary tract, in particular the bladder;
the upper aerodigestive tract and salivary glands, such as neuroendocrine cancers of the lung, in particular a small cell lung cancer, and such as neuroendocrine cancers of the thymus;
the thyroid, such as medullary thyroid carcinoma (MTC);
the adrenal gland, such as pheochromocytoma;
the skin, such as Merkel cell carcinoma (MCC);
the female reproductive organs, such as of the endometrium, the uterine cervix or the ovary;
the male reproductive organs, such as of the prostate or the testis; and
the head and neck.

In particular, the cancer may be selected from neuroendocrine cancers of the oesophagus, the stomach, the small intestine and the large intestine, the anal region, the pancreas, the bladder, the lung, the female reproductive organs, the male reproductive organs, and of the head and neck. In particular, the cancer may be a neuroendocrine cancer of the lung, in particular a neuroendocrine small cell lung cancer, or a neuroendocrine cancer of the male reproductive organs, in particular a neuroendocrine prostate cancer. In some embodiments, the cancer is a neuroendocrine small cell lung cancer. In another embodiment, the cancer is neuroendocrine prostate cancer.

In some embodiments, the cancer is not a neuroendocrine small cell lung cancer. In another embodiment, the cancer is not neuroendocrine prostate cancer.

In a particular embodiment, the subject is a moderate or high carcinoembryonic antigen-related cell adhesion molecule expresser. In particular, the subject may be a moderate CEACAM5 expresser, that is a subject who has a CEACAM5 expression consisting of 2+ or 3+ intensity, measured by CEACAM5 immunohistochemistry, in at least 1% of the tumor cell population or consisting of 1+ intensity in at least 50% of the tumor cell population. In particular, the subject may be a high CEACAM5 expresser, that is a subject who has a CEACAM5 expression consisting of 2+ or 3+ intensity, measured by CEACAM5 immunohistochemistry, in over 50% of the tumor cell population.

In a particular embodiment, the antibody is conjugated or linked to at least one growth inhibitory agent. In particular, said growth inhibitory agent may be (N²′-deacetyl-N²′-(3-mercapto-1-oxopropyl)-maytansine) (DM1) or N²′-deacetyl-N²′-(4-methyl-4-mercapto-1-oxopentyl)-maytansine (DM4).

In a particular embodiment, the antibody is a hCEACAM5-antibody, which comprises a heavy chain consisting of SEQ ID NO: 8 and a light chain consisting of SEQ ID NO: 9, and which is covalently linked to N2′-deacetyl-N2′(4-methyl-4-mercapto-1-oxopentyl)-maytansine (DM4) via N-succinimidyl pyridyldithiobutyrate (SPDB). In particular, the antibody may be Tusamitamab ravtansine.

In a particular embodiment, the antibody or immunoconjugate is administered at a dose level of 5, 10, 20, 30, 40, 60, 80, 100, 120, 150, 180, or 210 mg/m2 based on the body surface area of the subject. In a particular embodiment, the antibody or immunoconjugate is administered every 2 weeks, or every 3 weeks. In a particular embodiment, the antibody or immunoconjugate is administered at a dose level of 100 mg/m2 based on the body surface area of the subject every 2 weeks. In a particular embodiment, the antibody or immunoconjugate is administered at a dose of from about 100 mg/m² to about 200 mg/m² as a loading dose, in particular of about 135 mg/m², about 150 mg/m² or about 170 mg/m², as a loading dose.

### BRIEF DESCRIPTION OF THE FIGURES

FIG. 1 shows expression of CEACAM5 measured by RNA in PDx models. The abscissa axis represents each of the PDx models and the ordinate axis represents the expression level of CEACAM5 in terms of FPKM (Fragments per kilo base of transcript per million mapped fragments).

FIG. 2 represents the evolution of the tumor volume (in mm³) of different mouse models in the absence of any administration (control) (curve with squares), after administration of an irrelevant DM4-ADC immunoconjugate at a concentration of 35 mg/kg (curve with upright triangles) or of Tusamitamab ravtansine (also named SAR408701 in the figures) at a concentration of 5 mg/kg (curve with upside down triangles) in different mouse models over time (in days) (n=7 or 8). A) in model MR-0009-RO (NE+/CEACAM+ phenotype); B) in model MR-0084 (NE+/CEACAM5 focal phenotype); C) in model MR-0123 (ADK+/CEACAM− phenotype); D) in model MR-0191 (NE+/CEACAM+ phenotype) and E) in model MR-059 (NE+/CEACAM5+ phenotype). *, **, *** and *** showing the significance of the results.

FIG. 3 represents the evolution of the body weight (in g) of different mouse models in the absence of any administration (control) (curve with squares), after administration of an irrelevant DM4-ADC immunoconjugate at a concentration of 35 mg/kg (curve with upright triangles) or of Tusamitamab ravtansine (also named SAR408701 in the figures) at a concentration of 5 mg/kg (curve with upside down triangles) in different mouse models over time (in days) (n=7 or 8). A) in model MR-0009-RO; B) in model MR-0084; C) in model MR-0123; D) in model MR-0191 and E) in model MR-059.

FIG. 4 (A and B) illustrates the correlation between CEACAM5 expression (measured by FPKM from RNA sequencing) and NEPC markers (measured by FPKM from RNA sequencing) such as AR (Pearson r=−0.4214, p-value<0.0001), SYP (Pearson r=0.2617, p-value=0.0085), KLK3 (Pearson r=−0.4976, p-value<0.0001), ASCL1 (Pearson r=0.6352, p-value<0.0001), FOLH1 (Pearson r=−0.2584, p-value=0.0094) and DLL3 (Pearson r=0.5486, p-value<0.0001).

### DETAILED DESCRIPTION

The disclosure provides pharmaceutical compositions and methods of using these compositions for the treatment of neuroendocrine cancers which express CEACAM5, and the improvement of at least one symptom of the diseases. These compositions include at least one antibody that specifically binds CEACAM5, for example the antibody is antibody huMAb2-3. The ADC huMAb2-3-SPDB-DM4 is an immunoconjugate combining the huMAb2-3 (anti-CEACAM5) antibody and the maytansinoid derivative 4 (DM4), a potent antimitotic agent that inhibits microtubule assembly. DM4 is covalently bound to huMAb2-3 through an optimized linker SPDB [N-succinimidyl 4-(2-pyridyldithio)-butyrate] that is stable in plasma and cleavable inside cells. After binding and internalization in targeted cancer cells, huMAb2-3-SPDB-DM4 (Tusamitamab ravtansine) is degraded, releasing cytotoxic DM4 metabolites.

The inventors have found that many neuroendocrine cancers express the cell surface protein CEACAM5.

As can been seen in the experimental data provided further in the text, proof of concept was achieved in PDx models of neuroendocrine prostate cancer treated with tusamitamab ravtansine where it was shown that tumor volume significantly decreased in all models and that no significant toxicity was observed due to treatment. In particular, expression of CEACAM5 was shown to be correlated with expression of neuroendocrine prostate cancer markers.

Proof of concept was further achieved by the inventors in a subset of neuroendocrine small cell lung cancers treated with tusamitamab ravtansine. In particular, these data support the conclusion that tusamitamab ravtansine is effective in treating neuroendocrine SCLC.

These data support the conclusion that tusamitamab ravtansine is particularly effective in treating high CEACAM5 expressing neuroendocrine cancers.

### Definitions

As used herein, the term “about” in quantitative terms refers to plus or minus 10% of the value it modifies (rounded up to the nearest whole number if the value is not sub-dividable, such as a number of molecules or nucleotides). For example, the phrase “about 100 mg” would encompass 90 mg to 110 mg, inclusive; the phrase “about 2500 mg” would encompass 2250 mg to 2750 mg. When applied to a percentage, the term “about” refers to plus or minus 10% relative to that percentage. For example, the phrase “about 20%” would encompass 18-22% and “about 80%” would encompass 72-88%, inclusive. Moreover, where “about” is used herein in conjunction with a quantitative term it is understood that in addition to the value plus or minus 10%, the exact value of the quantitative term is also contemplated and described. For example, the term “about 23%” expressly contemplates, describes, and includes exactly 23%.

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic characteristic(s) of the disclosure. It is understood that the different embodiments of the disclosure using the term “comprising” or equivalent cover the embodiments where this term is replaced with “comprising only”, “consisting of” or “consisting essentially of”.

It is understood that wherever aspects are described herein with the language “comprising,” otherwise analogous aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

An “antibody” may be a natural or conventional antibody in which two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties, such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs therefore refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e., to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. A human framework region is a framework region that is substantially identical (about 85%, or more, in particular 90%, 95%, 97%, 99% or 100%) to the framework region of a naturally occurring human antibody.

In the context of the disclosure, CDR/FR definition in an immunoglobulin light or heavy chain is to be determined based on IMGT definition (Lefranc et al. Dev. Comp. Immunol., 2003, 27(1):55-77; www.imgt.org).

As used herein, the term “antibody” denotes conventional antibodies and fragments thereof, as well as single domain antibodies and fragments thereof, in particular variable heavy chain of single domain antibodies, and chimeric, humanized, bispecific or multispecific antibodies.

As used herein, antibody or immunoglobulin also includes “single domain antibodies” which have been more recently described and which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered single domain antibodies. Single domain antibodies may be derived from any species including, but not limited to mouse, human, camel, llama, goat, rabbit, bovine. Single domain antibodies may be naturally occurring single domain antibodies known as heavy chain antibody devoid of light chains. In particular, camelidiae species, for example camel, dromedary, llama, alpaca and guanaco, produce heavy chain antibodies naturally devoid of light chain. Camelid heavy chain antibodies also lack the CH1 domain.

The variable heavy chain of these single domain antibodies devoid of light chains are known in the art as “VHH” or “Nanobodv®”. Similar to conventional VH domains, VHHs contain four FRs and three CDRs. VHH have advantages over conventional antibodies: they are about ten times smaller than IgG molecules, and as a consequence properly folded functional VHH can be produced by in vitro expression while achieving high yield. Furthermore, VHH are very stable, and resistant to the action of proteases. The properties and production of VHH have been reviewed by Harmsen and De Haard H J (Appl. Microbiol. Biotechnol. 2007 November; 77(1):13-22).

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced by a single clone of B cells or hybridoma, but may also be recombinant, i.e., produced by protein engineering.

The term “humanized antibody” refers to an antibody which is wholly or partially of non-human origin, and which has been modified to replace certain amino acids, in particular in the framework regions of the VH and VL domains, in order to avoid or minimize an immune response in humans. The constant domains of a humanized antibody are most of the time human CH and CL domains.

“Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 and antigen binding activity, in which about a half of the N-terminal side of the heavy chain and the entire light chain are bound together through a disulfide bond. It is usually obtained among fragments by treating IgG with a protease, papain.

The term “F(ab′)2” refers to an antibody fragment having a molecular weight of about 100,000 and antigen binding activity, which is slightly larger than 2 identical Fab fragments bound via a disulfide bond of the hinge region. It is usually obtained among fragments by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. The human scFv fragment of the disclosure includes CDRs that are held in appropriate conformation, in particular by using gene recombination techniques. Divalent and multivalent antibody fragments can form either spontaneously by association of monovalent scFvs, or can be generated by coupling monovalent scFvs by a peptide linker, such as divalent sc(Fv)2. “dsFv” is a VH::VL heterodimer stabilized by a disulphide bond. “(dsFv)2” denotes two dsFv coupled by a peptide linker.

The term “bispecific antibody” or “BsAb” denotes an antibody which combines the antigen-binding sites of two antibodies within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “multispecific antibody” denotes an antibody which combines the antigen-binding sites of two or more antibodies within a single molecule.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains of the same chain, the domains are forced to pair with the complementary domains of another chain and create two antigen-binding sites.

An amino acid sequence “at least 85% identical to a reference sequence” is a sequence having, on its entire length, 85%, or more, in particular 90%, 91%, 92%, 93%, 94%, 95%, 96%, 97%, 98% or 99% sequence identity with the entire length of the reference amino acid sequence.

A percentage of “sequence identity” between amino acid sequences may be determined by comparing the two sequences, optimally aligned over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g., using the algorithm of Needleman and Wunsch J. Mol. Biol. 48:443 (1970). The percentage of sequence identity can be readily determined for instance using the program Needle, with the BLOSUM62 matrix, and the following parameters gap-open=10, gap-extend=0.5.

A “conservative amino acid substitution” is one in which an amino acid residue is substituted by another amino acid residue having a side chain R group with similar chemical properties (e.g., charge, size or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups can also be defined on the basis of amino acid size.

By “purified” and “isolated” it is meant, when referring to a polypeptide (i.e., the antibody of the disclosure) or a nucleotide sequence, that the indicated molecule is present in the substantial absence of other biological macromolecules of the same type. The term “purified” as used herein in particular means at least 75%, 85%, 95%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

As used herein, the term “subject” denotes a mammal, such as a rodent, a feline, a canine, and a primate. In particular, a subject according to the disclosure is a human.

### Antibody-Drug Conjugate Comprising an Anti-CEACAM5-Antibody

The present disclosure relates to an antibody-drug conjugate (ADC) comprising an anti-CEACAM5-antibody for the treatment of a cancer selected from neuroendocrine cancers expressing hCEACAM5 in a subject in need thereof.

The antibody-drug conjugate typically comprises an anti-CEACAM5-antibody and at least one chemotherapeutic agent, such as a cell growth inhibitory agent. An antibody-drug conjugate (ADC) comprises an anti-CEACAM5-antibody conjugated to at least one chemotherapeutic agent. In particular, in the antibody-drug conjugate, the anti-CEACAM5-antibody is covalently attached via a cleavable or non-cleavable linker to the at least one chemotherapeutic agent.

### Anti-CEACAM5-Antibody

The present disclosure relates to the use of an antibody or an antigen-binding fragment thereof, which specifically binds CEACAM5 for treating neuroendocrine cancers expressing CEACAM5. In particular, the antibody, or an antigen-binding fragment thereof, binds hCEACAM5.

According to a first aspect, the present disclosure relates to an antibody, or an antigen-binding fragment thereof, or an immunoconjugate comprising the antibody or said antigen-binding fragment, for use in treating a cancer selected from neuroendocrine cancers expressing CEACAM5 in a subject in need thereof, wherein the antibody specifically binds CEACAM5, in particular hCEACAM5, and wherein the antibody comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

According to an embodiment, the antibody is a humanized anti-CEACAM5-antibody.

According to the disclosure, the anti-CEACAM5-antibody comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

In a particular embodiment, the VH domain comprises SEQ ID NO: 1 (EVQLQESGPGLVKPGGSLSLSCAASGFVFSSYDMSWVRQTPERGLEWVAYISSGGGITYAP STVKGRFTVSRDNAKNTLYLQMNSLTSEDTAVYYCAAHYFGSSGPFAYWGQGTLVTVSS).

In a particular embodiment, the VL domain comprises SEQ ID NO: 2 (DIQMTQSPASLSASVGDRVTITCRASENIFSYLAWYQQKPGKSPKLLVYNTRTLAEGVPSRF SGSGSGTDFSLTISSLQPEDFATYYCQHHYGTPFTFGSGTKLEIK).

In a further embodiment, the anti-CEACAM5-antibody comprises a variable domain of a heavy chain (VH) consisting of SEQ ID NO: 1.

In a further embodiment, the anti-CEACAM5-antibody comprises a variable domain of a light chain (VL) consisting of SEQ ID NO: 2.

In particular, the anti-CEACAM5-antibody comprises:

a variable domain of heavy chain consisting
of sequence
(SEQ ID NO: 1)
EVQLQESGPGLVKPGGSLSLSCAASGFVFSSYDMSWVRQTPERGLEWV
AYISSGGGITYAPSTVKGRFTVSRDNAKNTLYLQMNSLTSEDTAVYYC
AAHYFGSSGPFAYWGQGTLVTVSS,
and
a variable domain of light chain consisting
of sequence
(SEQ ID NO: 2)
DIQMTQSPASLSASVGDRVTITCRASENIFSYLAWYQQKPGKSPKLLV
YNTRTLAEGVPSFSGSGSGTDFSLTISSLQPEDFATYYCQHHYGTPFT
FGSGTKLEIK.

In a further embodiment, the anti-CEACAM5-antibody comprises a variable domain of a heavy chain (VH) having at least 90% identity to SEQ ID NO: 1, and a variable domain of a light chain (VL) having at least 90% identity to SEQ ID NO: 2, wherein HCDR1 consists of SEQ ID NO: 3, HCDR2 consists of SEQ ID NO: 4, HCDR3 consists of SEQ ID NO: 5, LCDR1 consists of SEQ ID NO: 6, LCDR2 consists of amino acid sequence NTR, and LCDR3 consists of SEQ ID NO: 7.

In a further embodiment, the anti-CEACAM5-antibody comprises a variable domain of a heavy chain (VH) having at least 92%, at least 95%, at least 98% identity to SEQ ID NO: 1, and a variable domain of a light chain (VL) having at least 92%, at least 95%, at least 98% identity to SEQ ID NO: 2, wherein HCDR1 consists of SEQ ID NO: 3, HCDR2 consists of SEQ ID NO: 4, HCDR3 consists of SEQ ID NO: 5, LCDR1 consists of SEQ ID NO: 6, LCDR2 consists of amino acid sequence NTR, and LCDR3 consists of SEQ ID NO: 7.

In a particular embodiment, the anti-CEACAM5-antibody comprises a heavy chain (HC) comprising SEQ ID NO: 8.

In a further embodiment, the anti-CEACAM5-antibody comprises a light chain (LC) comprising SEQ ID NO: 9.

In a further embodiment, the anti-CEACAM5-antibody comprises a heavy chain (HC) consisting of SEQ ID NO: 8.

In a further embodiment, the anti-CEACAM5-antibody comprises a light chain (LC) consisting of SEQ ID NO: 9.

In particular, the anti-CEACAM5-antibody comprises:

a heavy chain consisting of sequence
(SEQ ID NO: 8)
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG,
and
a light chain consisting of sequence
(SEQ ID NO: 9)
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In a further embodiment, the anti-CEACAM5-antibody comprises a heavy chain (HC) having at least 90% sequence identity to SEQ ID NO: 8 and a light chain (LC) having at least 90% sequence identity to SEQ ID NO: 9, wherein HCDR1 consists of SEQ ID NO: 3, HCDR2 consists of SEQ ID NO: 4, HCDR3 consists of SEQ ID NO: 5, LCDR1 consists of SEQ ID NO: 6, LCDR2 consists of amino acid sequence NTR, and LCDR3 consists of SEQ ID NO: 7.

In a further embodiment, the anti-CEACAM5-antibody comprises a heavy chain (HC) having at least 92%, at least 95%, at least 98% identity to SEQ ID NO: 8 and a light chain (LC) having at least 92%, at least 95%, at least 98% identity to SEQ ID NO: 9, wherein HCDR1 consists of SEQ ID NO: 3, HCDR2 consists of SEQ ID NO: 4, HCDR3 consists of SEQ ID NO: 5, LCDR1 consists of SEQ ID NO: 6, LCDR2− consists of amino acid sequence NTR, and LCDR3 consists of SEQ ID NO: 7.

The anti-CEACAM5-antibody may also be a single domain antibody or a fragment thereof. In particular, a single domain antibody fragment may consist of a variable heavy chain (VHH) which comprises the HCDR1, HCDR2 and HCDR3 of the antibodies as described above. The antibody may also be a heavy chain antibody, i.e., an antibody devoid of light chain, which may or may not contain a CH1 domain.

The single domain antibody or a fragment thereof may also comprise the framework regions of a camelid single domain antibody, and optionally the constant domain of a camelid single domain antibody.

The anti-CEACAM5-antibody may also be an antibody fragment, in particular a humanized antibody fragment, selected from the group consisting of Fv, Fab, F(ab′)2, Fab′, dsFv, (dsFv)2, scFv, sc(Fv)2, and diabodies.

The antibody may also be a bispecific or multispecific antibody formed from antibody fragments, at least one antibody fragment being an antibody fragment according to the disclosure. Multispecific antibodies are polyvalent protein complexes as described for instance in EP 2 050 764 A1 or US 2005/0003403 A1.

The anti-CEACAM5-antibody and fragments thereof can be produced by any technique well known in the art. In particular, said antibodies are produced by techniques as hereinafter described.

The anti-CEACAM5-antibody and fragments thereof can be used in an isolated (e.g., purified) from or contained in a vector, such as a membrane or lipid vesicle (e.g., a liposome).

The anti-CEACAM5-antibody and fragments thereof may be produced by any technique known in the art, such as, without limitation, any chemical, biological, genetic, or enzymatic technique, either alone or in combination.

Knowing the amino acid sequence of the desired sequence, one skilled in the art can readily produce the anti-CEACAM5-antibody and fragments thereof, by standard techniques for production of polypeptides. For instance, they can be synthesized using well-known solid phase method, in particular using a commercially available peptide synthesis apparatus (such as that made by Applied Biosystems, Foster City, California) and following the manufacturer's instructions. Alternatively, anti-CEACAM5-antibody and fragments thereof can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Anti-CEACAM5-antibody and fragments thereof are suitably separated from the culture medium by conventional immunoglobulin purification procedures such as, for example, protein A-Sepharose, hydroxyapatite chromatography, gel electrophoresis, dialysis, or affinity chromatography.

Methods for producing humanized antibodies based on conventional recombinant DNA and gene transfection techniques are well known in the art (See, e. g., Riechmann L. et al. 1988; Neuberger M S. Et al. 1985). Antibodies can be humanized using a variety of techniques known in the art including, for example, the technique disclosed in the application WO2009/032661, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. Et al. (1994)), and chain shuffling (U.S. Pat. No. 5,565,332). The general recombinant DNA technology for preparation of such antibodies is also known (see European Patent Application EP 125023 and International patent Application WO 96/02576).

The Fab of the anti-CEACAM5-antibody can be obtained by treating an antibody which specifically reacts with CEACAM5 with a protease, such as papain. Also, the Fab of the anti-CEACAM5-antibody can be produced by inserting DNA sequences encoding both chains of the Fab of the anti-CEACAM5-antibody into a vector for prokaryotic expression, or for eukaryotic expression, and introducing the vector into prokaryotic or eukaryotic cells (as appropriate) to express the Fab of the anti-CEACAM5-antibody.

The F(ab′)2 of the anti-CEACAM5-antibody can be obtained treating an antibody which specifically reacts with CEACAM5 with a protease, pepsin. Also, the F(ab′)2 of the anti-CEACAM5-antibody can be produced by binding Fab′ described below via a thioether bond or a disulfide bond.

The Fab′ of the anti-CEACAM5-antibody can be obtained treating F(ab′)2 which specifically reacts with CEACAM5 with a reducing agent, such as dithiothreitol. Also, the Fab′ of the anti-CEACAM5-antibody can be produced by inserting DNA sequences encoding Fab′ chains of the antibody into a vector for prokaryotic expression, or a vector for eukaryotic expression, and introducing the vector into prokaryotic or eukaryotic cells (as appropriate) to perform its expression.

The scFv of the anti-CEACAM5-antibody can be produced by taking sequences of the CDRs or VH and VL domains as previously described, constructing a DNA encoding an scFv fragment, inserting the DNA into a prokaryotic or eukaryotic expression vector, and then introducing the expression vector into prokaryotic or eukaryotic cells (as appropriate) to express the scFv. To generate a humanized scFv fragment, a well-known technology called CDR grafting may be used, which involves selecting the complementary determining regions (CDRs) according to the disclosure and grafting them onto a human scFv fragment framework of known three-dimensional structure (see, e. g., WO98/45322; WO 87/02671; U.S. Pat. Nos. 5,859,205; 5,585,089; 4,816,567; EP0173494).

In an embodiment, the anti-CEACAM5 antibody is Tusamitamab (CAS [2349294-95-5].

### Chemotherapeutic Agent

The present disclosure also includes cytotoxic conjugates, or immunoconjugates, or antibody-drug conjugates, or conjugates. As used herein, all these terms have the same meaning and are interchangeable.

The antibody-drug conjugate for the use according to the present disclosure typically comprises at least one chemotherapeutic agent. A chemotherapeutic agent as used herein refers to an agent that kills cells, including cancer cells. Such agents favorably stop cancer cells from dividing and growing and cause tumors to shrink in size. The expression “chemotherapeutic agent” is used herein interchangeably with the expressions “cytotoxic agent”, “growth inhibitory agent”, “cell growth inhibitory agent” or “cytostatic drug”.

Accordingly, the disclosure relates to “immunoconjugates” comprising an antibody of the disclosure conjugated or linked to at least one growth inhibitory agent. In a particular embodiment, the growth inhibitory agent is a cytotoxic agent or a radioactive isotope.

A “growth inhibitory agent”, or “anti-proliferative agent”, which can be used indifferently, refers to a compound or composition which inhibits growth of a cell, especially tumour cell, either in vitro or in vivo.

In a further embodiment, the chemotherapeutic agent is selected from the group consisting of radioisotopes, protein toxins, small molecule toxins, and combinations thereof.

Radioisotopes include radioactive isotopes suitable for treating cancer. Such radioisotopes generally emit mainly beta-radiation. In a further embodiment, the radioisotopes are selected from the group consisting of At²¹¹, Bi²¹², Er¹⁶⁹, I¹³¹, I¹²⁵, Y⁹⁰, In¹¹¹, P³², Re¹⁸⁶, Re¹⁸⁸, Sm¹⁵³, Sr⁸⁹, radioactive isotopes of Lu, and combinations thereof. In an embodiment, the radioactive isotope is alpha-emitter isotope, more specifically Th²²⁷, which emits alpha-radiation.

The immunoconjugates according to the present disclosure can be prepared as described in the application WO2004/091668.

In a further embodiment, the small molecule toxins are selected from anti-metabolites, DNA-alkylating agents, DNA-cross-linking agents, DNA-intercalating agents, anti-microtubule agents, topoisomerase inhibitors, and combinations thereof.

In a further embodiment, the anti-microtubule agent is selected from the group consisting of taxanes, vinca alkaloids, maytansinoids, colchicine, podophyllotoxin, gruseofulvin, and combinations thereof.

The term “cytotoxic agent” or “growth inhibitory agent” as used herein refers to a substance that inhibits or prevents the function of cells and/or causes destruction of cells. The term “cytotoxic agent” is intended to include chemotherapeutic agents, enzymes, antibiotics, and toxins such as small molecule toxins or enzymatically active toxins of bacterial, fungal, plant or animal origin, including fragments and/or variants thereof, and the various antitumor or anticancer agents disclosed below.

In a particular embodiment, the “cytotoxic agent” or “growth inhibitory agent” is selected from the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, topoisomerase inhibitors, DNA alkylating agents, anti-tubulin agents, and CC-1065 or CC-1065 analogs. In some embodiments, the topoisomerase inhibitors are not topoisomerase I inhibitors. In some embodiments, the topoisomerase inhibitors are topoisomerase II inhibitors.

In a particular embodiment, the growth inhibitory agent is not a topoisomerase inhibitor.

In a particular embodiment, the growth inhibitory agent is selected from the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, DNA alkylating agents, anti-tubulin agents, and CC-1065 or CC-1065 analogs.

An analog as understood herein is a compound that may differ chemically from another compound, but shares the same or similar functional properties, in particular the same or similar biological properties as said compound.

As used herein, “enzymatically active toxins” are a type of toxin produced by certain organisms, such as bacteria, fungi, plants, or animals, that function by interfering with essential biological processes within the cells or tissues of the organism being targeted, similar to the way enzymes act. The enzymatic activity of these toxins is often the key mechanism by which they cause harm to the host organism. Examples of enzymatically active toxins include the botulinum toxin, the diphtheria toxin, the cholera toxin, the shiga toxin and the tetanus toxin.

As used herein, “taxoids” or “taxanes” are two classes of derivatives from taxol that have been developed for their anticancer chemotherapeutic properties. Examples of such compounds include, but are not limited to, paclitaxel, docetaxel, cabazitaxel, paclitaxel pliglumex, paclitaxel docohexaenoic acid, paclitaxel trevatide, AI-850, milataxel and ANG1005.

As used herein, “vincas” or “vinca alkaloids” are a set of anti-mitotic and anti-microtubule alkaloid agents originally derived from vinca plants that block beta-tubulin polymerization in a dividing cell and as such have been used in chemotherapy for cancer. Examples of vincas include but are not limited to vinblastine, vincristine, vindesine, vinorelbine, vincaminol, vineridine, and vinburnine.

Pyrrolobenzodiazepines (PBDs) are naturally occurring anticancer drug molecules consisting of a central pyrrolo[2,1-c][1,4]benzodiazepine core. Examples of PBDs and PBDs derivatives include, but are not limited to, tomaymycin, diazepam, anthramycin, sibiromycin, and porothramycin.

Cryptophycins are a family of macrolide molecules that are potent cytotoxins and have been studied for potential antiproliferative properties useful in developing chemotherapy. Examples or cryptophycins and their derivatives include, but are not limited to, cryptophycin-1, cryptophycin-52 (LY355703), and cryptophycin-55.

As used herein, “leptomycin derivatives” refer to members of the leptomycin family as defined in Kalesse et al in Synthesis 2002, 8, 981-1003, and includes: leptomycins, such as leptomycin A and leptomycin B, callystatins, ratjadones such as ratjadone A and ratjadone B, anguinomycins such as anguinomycin A, B, C, D, kasusamycins, leptolstatin, leptofuranins, such as leptofuranin A, B, C, D.

As used herein, “dolastatins” and “auristatins” include linear and cyclic peptides that were initially isolated from D. auricularia. Among them, certain dolastatins have shown effective cytotoxic affects on tumorous cells. Examples of dolastatin and dolastatin derivatives include, but are not limited to, dolastatin-10, dolastatin-15, dolastatinol, auristatins, symplostatin 1, and symplostatin 5.

As used herein, a “prodrug” is a pharmacologically inactive medication or compound that, after intake, is metabolized into a pharmacologically active drug. Type I prodrugs are bioactivated inside the cells (intracellularly) while type II prodrugs are bioactivated outside cells (extracellularly), especially in digestive fluids or in the body's circulatory system, particularly in the blood. Examples of type I prodrugs include, but are not limited to, aciclovir, fluorouracil, cyclophosphamide, diethylstilbestrol diphosphate, L-DOPA, mercaptopurine, mitomycin, zidovudine, carbamazepine, captopril, carisoprodol, heroin, molsidomine, leflunomide, paliperidone, phenacetin, primidone, psilocybin, sulindac, and fursultiamine. Examples of type II prodrugs include, but are not limited to, loperamide oxide, oxyphenisatin, sulfasalazine, acetylsalicylate, bacampicillin, bambuterol, chloramphenicol succinate, dipivefrin, fosphenytoin, lisdexamfetamine, pralidoxime, ADEPTs, GDEPTs and VDEPTs.

Topoisomerases are enzymes that have evolved to resolve topological problems in DNA via transient breakage of one or both strands of DNA. Topoisomerases I catalyze changes in DNA topology via transient single-stranded breaks in DNA while topoisomerases II catalyze changes in DNA topology via transient double-stranded breaks in DNA. Both human topoisomerases I and topoisomerases II (both α and β isoforms) can be targeted in anticancer chemotherapy by topoisomerases inhibitors.

Examples of topoisomerases I according to the present text include, but are not limited to, camptothecin (CPT) and analogs thereof such as deruxtecan, topotecan, irinotecan, SN-38, silatecan, cositecan, exatecan, lurtotecan, gimatecan, belotecan and rubitecan.

Examples of topoisomerases II according to the present text include, but are not limited to, anthracyclines, etoposide, teniposide, doxorubicin, daunorubicin, epirubicin, idarubicin, mitoxantrone, ICRF-193, ICRF-187 (dexrazoxane), merbarone, and aclarubicin.

As used herein, “DNA alkylating agents” are a class of antineoplastic or anticancer drugs which act by inhibiting the transcription of DNA into RNA and thereby stopping the protein synthesis. Examples of such compounds include, but are not limited to, altretamine, bendamustine, busulfan, carmustine, chlorambucil, cyclophosphamide, dacarbazine, ifosfamide, lomustine, lurbinectedin, mechlorethamine, melphalan, procarbazine, streptozocin, temozolomide, thiotepa, trabectedin and platinum coordination complexes such as carboplatin, cisplatin and oxaliplatin.

Anti-tubulin agents constitute a large class of compounds with broad activity both in solid tumors and hematologic malignancies, due to the interference with microtubule dynamics. Examples of such compounds include, but are not limited to, taxans, vinca alkaloids, dolastatins, estramustine, maytansinoids, halichondrins, nocodazole, cryptophycins, colchicine and its analogues, hemiasterlins, podophyllotoxin, combretastatins, 2-methoxyoestradiol, 4-substituted methoxybenzoyl-aryl-thiazoles (SMART), phenylahistins, steganacins and curacins.

CC-1065 is a natural anti-tumor agent with a unique structure produced by Streptomyces zelensis. C-1065 analogs are a class of alkylating agents that are highly suitable for targeted tumor therapies. Examples of C-1065 analogs include, but are not limited to, adozelesin, carzelesin and bis-indolyl-(seco)-CBl3 derivatives.

In some embodiments, the cytotoxic agent is a taxoid, vincas, a maytansinoid or maytansinoid analog such as DM1 or DM4, a small drug, a tomaymycin or pyrrolobenzodiazepine derivative, a cryptophycin derivative, a leptomycin derivative, an auristatin or dolastatin analog, a prodrug, topoisomerase inhibitors, in particular topoisomerase II inhibitors, a DNA alkylating agent, an anti-tubulin agent, a CC-1065 or CC-1065 analog.

In some embodiment a cytotoxic agent may be a maytansinoid.

As used herein “maytansinoids” denotes maytansinoids and maytansinoid analogs. Maytansinoids are drugs that inhibit microtubule formation and that are highly toxic to mammalian cells.

Examples of suitable maytansinoids include maytansinol and maytansinol analogs, and combinations thereof.

Examples of suitable maytansinol analogues include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Specific examples of suitable analogues of maytansinol having a modified aromatic ring include:
(1) C-19-dechloro (U.S. Pat. No. 4,256,746) (prepared by LAH reduction of ansamytocin P2);
(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and
(3) C-20-demethoxy, C-20-acyloxy (—OCOR), +/−dechloro (U.S. Pat. No. 4,294,757) (prepared by acylation using acyl chlorides).

Specific examples of suitable analogues of maytansinol having modifications of other positions include:
(1) C-9-SH (U.S. Pat. No. 4,424,219) (prepared by the reaction of maytansinol with H₂S or P₂S₅);
(2) C-14-alkoxymethyl (dimethoxy/CH₂OR) (U.S. Pat. No. 4,331,598);
(3) C-14-hydroxymethyl or acyloxymethyl (CH₂OH or CH₂Oac) (U.S. Pat. No. 4,450,254) (prepared from Nocardia);
(4) C-15-hydroxy/acyloxy (U.S. Pat. No. 4,364,866) (prepared by the conversion of maytansinol by Streptomyces);
(5) C-15-methoxy (U.S. Pat. Nos. 4,313,946 and 4,315,929) (isolated from Trewia nudiflora);
(6) C-18-N-demethyl (U.S. Pat. Nos. 4,362,663 and 4,322,348) (prepared by the demethylation of maytansinol by Streptomyces); and
(7) 4,5-deoxy (U.S. Pat. No. 4,371,533) (prepared by the titanium trichloride/LAH reduction of maytansinol).

In an embodiment of the disclosure, the cytotoxic conjugates of the present disclosure utilize the thiol-containing maytansinoid (DM1), formally termed N²′-deacetyl-N²′-(3-mercapto-1-oxopropyl)-maytansine, as the cytotoxic agent. DM1 is represented by the following structural formula (I):

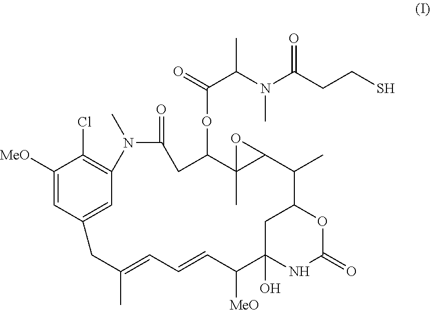 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another embodiment, the cytotoxic conjugates of the present disclosure utilize the thiol-containing maytansinoid DM4, formally termed N^{2′}-deacetyl-N-^{2′}(4-methyl-4-mercapto-1-oxopentyl)-maytansine, as the cytotoxic agent. DM4 is represented by the following structural formula (II):

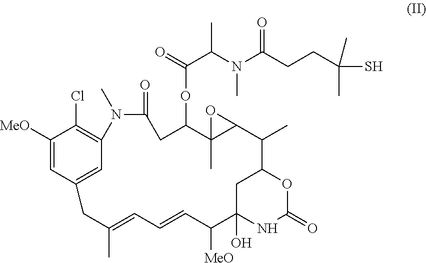 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In a particular embodiment, the inhibitory agent or growth inhibitor agent is (N2′-deacetyl-N2′-(3-mercapto-1-oxopropyl)-maytansine) DM1 or N2′-deacetyl-N-2′(4-methyl-4-mercapto-1-oxopentyl)-maytansine (DM4).

In further embodiments of the disclosure, other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 reagents and any aggregate which may be present in the solution.

Examples of these cytotoxic agents and of methods of conjugation are further given in the application WO 2008/010101 which is incorporated by reference.

Accordingly, in a further embodiment, the maytansinoids are selected from the group consisting of (N2′-deacetyl-N2′-(3-mercapto-1-oxopropyl)-maytansine) DM1 or N2′-deacetyl-N-2′(4-methyl-4-mercapto-1-oxopentyl)-maytansine (DM4), and combinations thereof.

In some embodiments, the antibodies of the present disclosure are covalently attached, directly or via a cleavable or non-cleavable linker, to at least one growth inhibitory agent or cytotoxic agent.

In particular, the antibody is covalently attached via a cleavable or non-cleavable linker to the at least one growth inhibitory agent.

“Linker”, as used herein, means a chemical moiety comprising a covalent bond or a chain of atoms that covalently attaches a polypeptide to a drug moiety.

The conjugates may be prepared by in vitro methods. In order to link a drug or prodrug to the antibody, a linking group is used. Suitable linking groups are well known in the art and include disulfide groups, thioether groups, acid labile groups, photolabile groups, peptidase labile groups and esterase labile groups. Conjugation of an antibody of the disclosure with cytotoxic agents or growth inhibitory agents may be made using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to the antibody (WO 94/11026).

The linker may be a “cleavable linker” facilitating release of the chemotherapeutic agent in the cell. For example, an acid-labile linker, a peptidase-sensitive linker, an esterase labile linker, a photolabile linker or a disulfide-containing linker (See e.g., U.S. Pat. No. 5,208,020) may be used. The linker may be also a “non-cleavable linker” (for example SMCC linker) that might led to better tolerance in some cases.

In a particular embodiment, the linker is selected from the group consisting of N-succinimidyl pyridyldithiobutyrate (SPDB), 4-(Pyridin-2-yldisulfanyl)-2-sulfo-butyric acid (sulfo-SPDB), and succinimidyl (N-maleimidomethyl) cyclohexane-1-carboxylate (SMCC).

In a further embodiment, the linker binds to a lysine or cysteine residue in the Fc region of the anti-CEACAM5 antibody. In a further embodiment, the linker forms a disulfide bond or a thioether bond with the maytansine.

The linker may be a “cleavable linker” facilitating release of the cytotoxic agent or growth inhibitory agent in the cell. For example, an acid-labile linker, a peptidase-sensitive linker, an esterase labile linker, a photolabile linker or a disulfide-containing linker (See e.g. U.S. Pat. No. 5,208,020) may be used. The linker may be also a “non-cleavable linker” (for example SMCC linker) that might lead to better tolerance in some cases.

Alternatively, a fusion protein comprising the antibody of the disclosure and a cytotoxic or growth inhibitory polypeptide may be made, by recombinant techniques or peptide synthesis. The length of DNA may comprise respective regions encoding the two portions of the conjugate either adjacent one another or separated by a region encoding a linker peptide which does not destroy the desired properties of the conjugate.

The antibodies of the present disclosure may also be used in Dependent Enzyme Mediated Prodrug Therapy by conjugating the polypeptide to a prodrug-activating enzyme which converts a prodrug (e.g. a peptidyl chemotherapeutic agent, see WO81/01145) to an active anti-cancer drug (See, for example, WO 88/07378 and U.S. Pat. No. 4,975,278). The enzyme component of the immunoconjugate useful for ADEPT includes any enzyme capable of acting on a prodrug in such a way so as to convert it into its more active, cytotoxic form. Enzymes that are useful in the method of this disclosure include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anticancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. The enzymes can be covalently bound to the polypeptides of the disclosure by techniques well known in the art such as the use of the heterobifunctional crosslinking reagents discussed above.

According to an embodiment, in the conjugate of the disclosure, the growth inhibitory agent is a maytansinoid, in an embodiment DM1 or DM4.

In said conjugate, the antibody is conjugated to said at least one growth inhibitory agent by a linking group. In an embodiment said linking group is a cleavable or a non-cleavable linker, such as SPDB, sulfo-SPDB, or SMCC.

In particular, the anti-CEACAM5-antibody-drug conjugate may be selected from the group consisting of:
i) the anti-CEACAM5-SPDB-DM4 conjugate of formula (III):

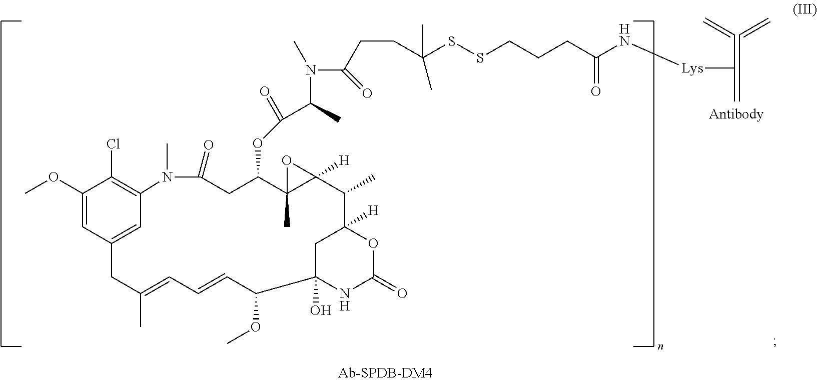

ii) the anti-CEACAM5-sulfo-SPDB-DM4 conjugate of formula (IV):
and

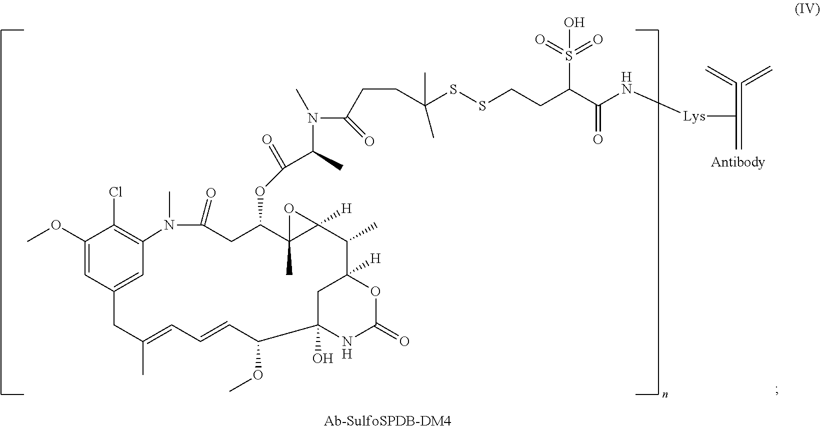

iii) the anti-CEACAM5-SMCC-DM1 conjugate of formula (V):

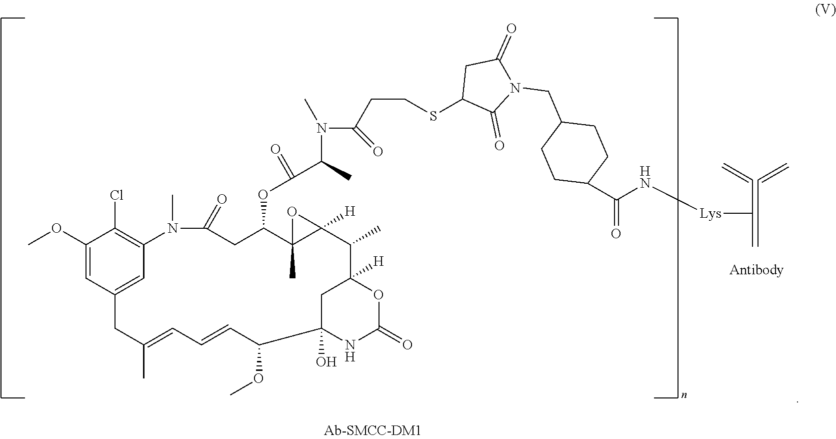

In an embodiment the immunoconjugate is a immunoconjugate of formula (III), (IV) or (V) as defined above, in which the antibody is an antibody described herein.

In a particular embodiment, the immunoconjugate comprises a hCEACAM5-antibody, which comprises a heavy chain (VH) consisting of SEQ ID NO: 8 and a light chain (VL) consisting of SEQ ID NO: 9, and which is covalently linked to N²′-deacetyl-N²′(4-methyl-4-mercapto-1-oxopentyl)-maytansine (DM4) via N-succinimidyl pyridyldithio-butyrate (SPDB).

In a particular embodiment, the immunoconjugate is Tusamitamab ravtansine (CAS [2254086-60-5]).

In general, the immunoconjugate can be obtained by a process comprising the steps of:
(i) bringing into contact an optionally-buffered aqueous solution of a cell-binding agent (e.g. an antibody according to the disclosure) with solutions of a linker and a cytotoxic compound;
(ii) then optionally separating the conjugate which was formed in (i) from the unreacted cell-binding agent.

The aqueous solution of cell-binding agent can be buffered with buffers such as, e.g. potassium phosphate, acetate, citrate or N-2-Hydroxyethylpiperazine-N′-2-ethanesulfonic acid (Hepes buffer). The buffer depends upon the nature of the cell-binding agent. The cytotoxic compound is in solution in an organic polar solvent, e.g. dimethyl sulfoxide (DMSO) or dimethylacetamide (DMA).

The reaction temperature is usually comprised between 2° and 40° C. The reaction time can vary from 1 to 24 hours. The reaction between the cell-binding agent and the cytotoxic agent can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC). Purification by dialysis or diafiltration can also be used.

As used herein, the term “aggregates” means the associations which can be formed between two or more cell-binding agents, said agents being modified or not by conjugation. The aggregates can be formed under the influence of a great number of parameters, such as a high concentration of cell-binding agent in the solution, the pH of the solution, high shearing forces, the number of bonded dimers and their hydrophobic character, the temperature (see Wang & Gosh, 2008, J. Membrane Sci., 318: 311-316, and references cited therein); note that the relative influence of some of these parameters is not clearly established. In the case of proteins and antibodies, the person skilled in the art will refer to Cromwell et al. (2006, AAPS Journal, 8(3): E572-E579). The content in aggregates can be determined with techniques well known to the skilled person, such as SEC (see Walter et al., 1993, Anal. Biochem., 212(2): 469-480).

After step (i) or (ii), the conjugate-containing solution can be submitted to an additional step (iii) of chromatography, ultrafiltration and/or diafiltration.

The conjugate is recovered at the end of these steps in an aqueous solution.

According to an embodiment, the conjugate according to the disclosure is characterised by a “drug-to-antibody ratio” (or “DAR”) ranging from 1 to 10, for instance from 2 to 5, in particular from 3 to 4. This is generally the case of conjugates including maytansinoid molecules.

This DAR number can vary with the nature of the antibody and of the drug (i.e. the growth-inhibitory agent) used along with the experimental conditions used for the conjugation (like the ratio growth-inhibitory agent/antibody, the reaction time, the nature of the solvent and of the cosolvent if any). Thus, the contact between the antibody and the growth-inhibitory agent leads to a mixture comprising several conjugates differing from one another by different drug-to-antibody ratios; optionally the naked antibody; optionally aggregates. The DAR that is determined is thus a mean value.

A method which can be used to determine the DAR consists in measuring spectrophotometrically the ratio of the absorbance at of a solution of substantially purified conjugate at λ_{D} and 280 nm. 280 nm is a wavelength generally used for measuring protein concentration, such as antibody concentration. The wavelength λ_{D} is selected so as to allow discriminating the drug from the antibody, i.e., as readily known to the skilled person, λ_{D} is a wavelength at which the drug has a high absorbance and λ_{D} is sufficiently remote from 280 nm to avoid substantial overlap in the absorbance peaks of the drug and antibody. Λ_{D} may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science:

The absorbances for the conjugate at λ_{D} (A_{λD}) and at 280 nm (A₂₈₀) are measured either on the monomeric peak of the size exclusion chromatography (SEC) analysis (allowing to calculate the “DAR(SEC)” parameter) or using a classic spectrophotometer apparatus (allowing to calculate the “DAR(UV)” parameter). The absorbances can be expressed as follows:

$${A}_{λ} D = ( cD \times ε {D}_{λ} D ) + ( cA \times ε {A}_{λ} D )$$

$${A}_{280} = ( cD \times ε {D}_{280} ) + ( cA \times ε {A}_{280} )$$

wherein:
cD and cA are respectively the concentrations in the solution of the drug (i.e., chemotherapeutic agent) and of the antibody
εD_{λ}D and εD₂₈₀ are respectively the molar extinction coefficients of the drug at λD and 280 nm
εA_{λ}D and εA₂₈₀ are respectively the molar extinction coefficients of the antibody at D and 280 nm.

Resolution of these two equations with two unknowns leads to the following equations:

$$cD = [ ( ε {A}_{280} \times {A}_{λ} D ) - ( ε {A}_{λ} D \times {A}_{280} ) ] / [ ( ε {D}_{λ} D \times ε {A}_{280} ) - ( ε {A}_{λ} D \times ε {D}_{280} ) ]$$

$$cA = [ {A}_{280} - ( cD \times ε {D}_{280} ) ] / ε {A}_{280}$$

The average DAR is then calculated from the ratio of the drug concentration to that of the antibody: DAR=cD/cA.

In a particular embodiment, the immunoconjugate comprises a hCEACAM5-antibody, which comprises a heavy chain consisting of SEQ ID NO: 8 and a light chain consisting of SEQ ID NO: 9, and which is covalently linked to N²′-deacetyl-N²′(4-methyl-4-mercapto-1-oxopentyl)-maytansine (DM4) via N-succinimidyl pyridyldithiobutyrate (SPDB).

In a particular embodiment, the immunoconjugate is Tusamitamab ravtansine.

### Neuroendocrine Cancers

The present disclosure relates to an antibody, or an antigen-binding fragment thereof, or an immunoconjugate comprising said antibody or said antigen-binding fragment, for use in treating a cancer selected from neuroendocrine cancers expressing CEACAM5 in a subject in need thereof.

It further relates to a method for treating cancers selected from neuroendocrine cancers expressing hCEACAM5 in a subject in need thereof, the method comprising administering an antibody, or an immunoconjugate comprising the antibody.

Neuroendocrine cancers or carcinomas are neoplasms that arise from cells of the endocrine and nervous systems. Two main histological subtypes of neuroendocrine cancers have been identified: the small cell type and the large cell type. Neuroendocrine carcinomas may occur in almost any body site. Lung small cell neuroendocrine carcinoma is by far the most frequent subset; the others are rare or very rare. Neuroendocrine carcinomas are usually diagnosed at an advanced, metastatic stage.

Selectively targeting potent cytotoxic agents to tumor cells using ADCs has now been shown to be an effective strategy for the treatment of cancer, as demonstrated by the recent approvals of brentuximab vedotin for the treatment of Hodgkin lymphoma and trastuzumab emtansine (T-DM1) for the treatment of relapsed metastatic HER2+ breast cancer. Many other malignant diseases with unmet medical needs could benefit from such therapeutic options. The mechanism of action of ADCs begins with its binding to a specific antigen, sufficiently expressed on the tumor cells in order to achieve a selective and efficient internalization of the drug.

A neuroendocrine cancer of the disclosure is selected from neuroendocrine tumors (NET), neuroendocrine carcinomas (NEC), Mixed Neuroendocrine-Non-Neuroendocrine Neoplasma (MiNEN), Pheochromocytoma and Medullary thyroid carcinoma (MTC).

In a particular embodiment, the cancer is selected from neuroendocrine cancers of:
the gastrointestinal and pancreatobiliary tract, such as neuroendocrine cancers of the digestive tract in particular the oesophagus, the stomach, the pancreas, the liver, the small intestine and large intestine or the anal region, or such as neuroendocrine cancers of the urinary tract, in particular the bladder;
the upper aerodigestive tract and salivary glands, such as neuroendocrine cancers of the lung, in particular a small cell lung cancer, and such as neuroendocrine cancers of the thymus;
the thyroid such as medullary thyroid carcinoma (MTC);
the adrenal gland such as pheochromocytoma;
the skin such as Merkel cell carcinoma (MCC);
the female reproductive organs, such as of the endometrium, the uterine cervix or the ovary;
the male reproductive organs, such as of the prostate or the testis, in particular the testis, and
the head and neck.

In a particular embodiment, the neuroendocrine cancer is selected from neuroendocrine cancers of the oesophagus, the stomach, the small intestine and the large intestine, the anal region, the pancreas, the bladder, the lung, the female reproductive organs, the male reproductive organs and of head and neck.

In a particular embodiment, the neuroendocrine cancer is a neuroendocrine cancer of the lung, in particular a neuroendocrine small cell lung cancer, or a neuroendocrine cancer of the male reproductive organs, in particular a neuroendocrine prostate cancer.

In a particular embodiment, the neuroendocrine cancer is a neuroendocrine cancer of the lung, in particular a neuroendocrine small cell lung cancer.

In a particular embodiment, the neuroendocrine cancer is not a neuroendocrine small cell lung cancer. In particular, the neuroendocrine cancer is selected from the group consisting of neuroendocrine cancers of the oesophagus, the stomach, the small intestine and the large intestine, the anal region, the pancreas, the bladder, the female reproductive organs, the male reproductive organs, the thyroid and the head and neck.

In a particular embodiment, the neuroendocrine cancer is a neuroendocrine prostate cancer (NEPC).

In a particular embodiment, the neuroendocrine cancer is not prostate cancer. In particular, the neuroendocrine cancer is not NEPC. In particular, the neuroendocrine cancer is selected from the group consisting of neuroendocrine cancers of the oesophagus, the stomach, the small intestine and the large intestine, the anal region, the pancreas, the bladder, the lung, the female reproductive organs, the thyroid and the head and neck.

Neuroendocrine subjects to be treated according to the disclosure may be classified as moderate or high CEACAM5 expressers.

In an embodiment, the subject is a moderate or high carcinoembryonic antigen-related cell adhesion molecule expresser.

In certain embodiments, the subject is a moderate CEACAM5 expresser. Moderate CEACAM5 expressers have a CEACAM5 expression consisting of +2 or 3+ intensity in at least 1% of the tumor cell population or consisting of 1+ intensity in at least 50% of the tumor cell population, measured by hCEACAM5 immunohistochemistry.

In certain embodiments, the subject is a high CEACAM5 expresser. High CEACAM5 expressers have a CEACAM5 expression consisting of 2+ or 3+ intensity in over 50% of the tumor cell population, measured by hCEACAM5 immunohistochemistry.

In a particular embodiment, the disclosure further relates to an antibody, or an antigen-binding fragment thereof, or an immunoconjugate comprising said antibody or said antigen-binding fragment, for use as a medicament.

In a particular embodiment, the disclosure relates to the use of an antibody, or an antigen-binding fragment thereof, or an immunoconjugate comprising said antibody or said antigen-binding fragment, for the manufacture of a medicament for the treatment of a cancer selected from neuroendocrine cancers expressing CEACAM5 in a subject in need thereof.

Said neuroendocrine cancers may be selected from any of those previously described in the disclosure.

In a particular embodiment, the disclosure relates to an immunoconjugate comprising an antibody or an antigen-binding fragment thereof, for use in treating a cancer selected from neuroendocrine cancers expressing hCEACAM5 in a subject in need thereof, wherein the antibody specifically binds hCEACAM5 and wherein the antibody, or the antigen-binding fragment thereof, comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT)
   wherein the antibody is conjugated or linked to at least one growth inhibitory agent, said at least one growth inhibitory agent being selected from the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, topoisomerase II inhibitors, DNA alkylating agents, anti-tubulin agents, and CC-1065 or CC-1065 analogs, in particular is not a topoisomerase I inhibitor.

In a particular embodiment, the disclosure relates to an immunoconjugate comprising an antibody or an antigen-binding fragment thereof, for use in treating a cancer selected from neuroendocrine cancers expressing hCEACAM5 in a subject in need thereof, wherein the antibody specifically binds hCEACAM5 and wherein the antibody, or the antigen-binding fragment thereof, comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT)
   wherein the antibody is conjugated or linked to at least one growth inhibitory agent, said at least one growth inhibitory agent being selected from the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, topoisomerase inhibitors, DNA alkylating agents, anti-tubulin agents, and CC-1065 or CC-1065 analogs,
   wherein the neuroendocrine cancer is selected from the group consisting of neuroendocrine cancers of the oesophagus, the stomach, the small intestine and the large intestine, the anal region, the pancreas, the bladder, the female reproductive organs, the male reproductive organs, the thyroid and the head and neck, in particular said neuroendocrine cancer is not a neuroendocrine small cell lung carcinoma (SCLC).

### Pharmaceutical Compositions

The antibodies or immunoconjugates of the disclosure may be combined with pharmaceutically acceptable excipients, and optionally sustained-release matrices, such as biodegradable polymers, to form therapeutic compositions.

Thus, another object of the disclosure relates to a pharmaceutical composition comprising an antibody or an immunoconjugate of the disclosure and a pharmaceutically acceptable carrier or excipient.

The disclosure also relates to a polypeptide or an immunoconjugate according to the disclosure, for use as a medicament.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, buffer phosphate, acetate, citrate, succinate; amino acids and derivates such as histidine, arginine, glycine, proline, glycylglycine; inorganic salts NaCl, calcium chloride; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an antioxidant such as tryptamine and a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the disclosure can be formulated for a topical, oral, parenteral, intranasal, intravenous, intramuscular, subcutaneous or intraocular administration and the like.

The therapeutic compositions of the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA, incorporated herein by reference in its entirety. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311, incorporated herein by reference in its entirety.

Various delivery systems are known and can be used to administer the pharmaceutical composition of the disclosure, e.g., encapsulation in liposomes, microparticles, microcapsules, receptor mediated endocytosis (see, e.g., Wu et al. (1987) J. Biol. Chem. 262:4429-4432, incorporated herein by reference in its entirety). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The CEACAM5 antibody, or an immunoconjugate comprising the antibody, can be administered subcutaneously.

The pharmaceutical composition can also be delivered in a vesicle, such as a liposome (see Langer (1990) Science 249:1527-1533, incorporated herein by reference in its entirety). In certain situations, the pharmaceutical composition can be delivered in a controlled release system, for example, with the use of a pump or polymeric materials. In another embodiment, a controlled release system can be placed in proximity of the composition's target, thus requiring only a fraction of the systemic dose.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc.). As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In an embodiment, the pharmaceutical compositions contain vehicles which are pharmaceutically acceptable for a formulation capable of being injected. These may be isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administrated through drug combination devices.

The doses used for the administration can be adapted as a function of various parameters, and for instance as a function of the mode of administration used, of the relevant pathology, or alternatively of the desired duration of treatment.

To prepare pharmaceutical compositions, an effective amount of the antibody or immunoconjugate of the disclosure may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and injectable with the appropriate device or system for delivery without degradation. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

An antibody or immunoconjugate of the disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with any of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more concentrated, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject.

The antibody or immunoconjugate of the disclosure may be formulated within a therapeutic mixture to comprise about 0.01 to 100 milligrams, per dose or so.

In addition to the antibody or immunoconjugate formulated for parenteral administration, such as intravenous or intramuscular injection, other pharmaceutically acceptable forms include, e.g., tablets or other solids for oral administration; time release capsules; and any other form currently used.

In certain embodiments, the use of liposomes and/or nanoparticles is contemplated for the introduction of polypeptides into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles, or biodegradable polylactide or polylactide co glycolide nanoparticles that meet these requirements are contemplated for use in the present disclosure, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

### Methods of Administration

The methods described herein comprise administering a therapeutically effective amount of an anti-CEACAM5 antibody, or an immunoconjugate comprising the antibody, to a subject.

As used herein, an “effective amount” or “therapeutically effective amount” is a dose of the therapeutic that results in treatment of neuroendocrine cancer. As used herein, “treating” refers to causing a detectable improvement in one or more symptoms associated with neuroendocrine cancer or causing a biological effect (e.g., a decrease in the level of a particular biomarker) that is correlated with the underlying pathologic mechanism(s) giving rise to the condition or symptom(s). For example, a dose of anti-CEACAM5 antibody, or an immunoconjugate comprising the antibody, which causes an improvement in any of the symptoms or conditions associated with neuroendocrine cancers is deemed a “therapeutically effective amount”:

In another example, a treatment has not been effective when a dose of anti-CEACAM5 antibody, or an immunoconjugate comprising the antibody, does not result in a detectable improvement in one or more parameters or symptoms associated with neuroendocrine cancer or which does not cause a biological effect that is correlated with the underlying pathologic mechanism(s) giving rise to the condition or symptom(s) of such cancers.

According to some of these embodiments, the anti-CEACAM5 antibody, or an immunoconjugate comprising the antibody, is administered intravenously.

In accordance with the methods of the present disclosure, a therapeutically effective amount of anti-CEACAM5 antibody, or an immunoconjugate comprising the antibody, that is administered to the subject will vary depending upon the age and the size (e.g., body weight or body surface area) of the subject as well as the route of administration and other factors well known to those of ordinary skill in the art.

A treatment, or course of treatment, may comprise at least one cycle of treatment.

In some embodiments, a treatment may comprise a first cycle of treatment, i.e., cycle 1, and at least one additional cycle of treatment, i.e., cycle(s) 2, 3, 4, 5, 6, 7, 8, 9, 10, 11, 12, 13, 14, 15, 16, or more.

The first cycle and the additional cycle(s) may be identical or different.

For example, the first cycle may comprise an administration of a loading dose, and the additional cycle(s) may comprise an administration of a maintenance dose.

Alternatively, the first and additional cycles may comprise an administration of a same dose.

In some embodiments, the antibody-drug conjugate comprising an anti-CEACAM5-antibody may be administered at a loading dose in a first cycle and at a maintenance dose in additional cycle(s).

In some embodiments, the antibody-drug conjugate comprising an anti-CEACAM5-antibody may be administered at a same dose in a first cycle and additional cycle(s).

A cycle of treatment may last from about 1 to about 6 weeks, from 1 to 4 weeks, from 1 to 3 weeks.

In some embodiments, a cycle of treatment may last at least about two weeks.

In some embodiments, a cycle of treatment may last at least about three weeks.

In some embodiment, a cycle of treatment may comprise a period of treatment at least on day 1, for example on day 1, 2, 3, 4, 5 or 6, of the cycle and a period of rest lasting until the completion of said cycle. The periods of treatment and the periods of rest may be identical or different between a first cycle and an at least one additional cycle. In some embodiments, the periods of treatment and the periods of rest may be identical between a first cycle and an at least one additional cycle.

In some embodiment, a cycle of treatment may comprise a period of treatment on day 1 of the cycle and a period of rest lasting until the completion of said cycle.

A treatment (or course of treatment) may comprise at least a first cycle (cycle 1) of treatment and at least one additional (subsequent) cycle. A treatment may comprise from 2 to 16, from 3 to 15, from 4 to 14, from 5 to 13, from 6 to 12, from 7 to 11, from 8 to 10, or about 9 cycles. A treatment may comprise 2, 3, 4, 5, 6, 7, 8, 9, 10, 11, 12, 13, 14, 15, 16 or more cycles.

In certain embodiments, the dose of the antibody, or an immunoconjugate comprising the antibody, varies depending on the body surface area of the subject. In certain embodiments, the dose of anti-CEACAM5 antibody, or an immunoconjugate comprising the antibody, administered to the subject is from about 1 mg/m² to about 500 mg/m². In some embodiments, the dose of the antibody, or an immunoconjugate comprising the antibody, administered to the subject is from about 5 mg to about 300 mg/m². In various embodiments, the dose of the antibody or an immunoconjugate comprising the antibody, administered to the subject is from about 5 to about 250 mg/m².

In a particular embodiment, the antibody or immunoconjugate is administered at a dose level of 5, 10, 20, 30, 40, 60, 80, 100, 120, 150, 180, or 210 mg/m² based on the body surface area of the subject. In various embodiments, the antibody, or an immunoconjugate comprising the antibody, is administered at a dose of about 2.5 mg/m² to about 5 mg/m². For example, the antibody, or an immunoconjugate comprising the antibody, is administered for a period of time (e.g., 30 minutes and one hour) at a dose of about 2.5 mg/m² to about 5 mg/m². The dose includes 2.5 mg/m² of the antibody, 5 mg/m² of the antibody, or an immunoconjugate comprising the antibody, and all doses in between 2.5 mg/m² and 5 mg/m², for example 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 mg/m².

For example, the present disclosure includes (but is not limited to) methods wherein about 1 mg/m², about 5 mg/m², 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 205 mg/m², about 210 mg/m², about 215 mg/m², about 220 mg/m², about 225 mg/m², about 230 mg/m², about 235 mg/m², about 240 mg/m², about 245 mg/m², about 250 mg/m², about 255 mg/m², about 260 mg/m², about 265 mg/m², about 270 mg/m², about 275 mg/m², about 280 mg/m², about 285 mg/m², about 290 mg/m², about 295 mg/m², about 300 mg/m², about 325 mg/m², about 350 mg/m², about 375 mg/m², about 400 mg/m², about 425 mg/m², about 450 mg/m², about 475 mg/m², or about 500 mg/m² of anti-CEACAM5 antibody, or an immunoconjugate comprising the antibody, is administered to the patient per week or once every two weeks.

In various embodiments, the antibody, or immunoconjugate, is administered at a dose level of 5, 10, 20, 30, 40, 60, 80, 100, 120, 150, 180, or 210 mg/m² based on the body surface area of the subject.

In a particular embodiment, the antibody or immunoconjugate is administered every 14 days (2 weeks), or every 3 weeks

In a particular embodiment, the antibody or immunoconjugate is administered at a dose level of 100 mg/m2 based on the body surface area of the subject every 2 weeks.

As used herein, “administered from about 1 to about 500 mg/kg” means that the referred to substance is administered at any value within the stated range including the endpoints of the range. For example, “the dose of anti-CEACAM5 antibody, or a immunoconjugate comprising the antibody, administered to the patient is from 1 mg/m² to 500 mg/m²,” includes administration of 1 mg/m² of the anti-CEACAM5 antibody, or of an immunoconjugate comprising the antibody, 500 mg/m² of the anti-CEACAM5 antibody, or of an immunoconjugate comprising the antibody, and all doses in between. In an embodiment, the CEACAM5 antibody, or an immunoconjugate comprising the antibody, is administered at a dose of about 5, 10, 20, 30, 40, 60, 80, 100, 120, 150, 180, or 210 mg/m² over a period of time, for example once every 14 days (i.e., every two weeks) or 3 weeks.

In various embodiments, the dose is administered at a constant rate. Alternatively, the dose is administered at a variable rate. In various embodiments, the dose is administered at a constant rate of about 0.5, 1, 1.5, 2, 2.5, 3, 3.5, 4, 2.5 or 5 mg/min. In various embodiments, the antibody, or an immunoconjugate comprising the antibody, is administered at a rate for the first 30 minutes or for the first 1 hour. In various embodiments, after about 30 minutes or 1 hour, the rate of administration of the antibody is changed. For example, the rate is decreased. In various embodiments, the rate is increased. In various embodiments, the antibody, or an immunoconjugate comprising the antibody, is administered at a rate of 2.5 mg/min for the first 30 minutes or 1 hour. In various embodiments, after about 30 minutes or 1 hour, the rate of administration of the antibody, or of an immunoconjugate comprising the antibody, is increased to 5 mg/min.

The methods of the present disclosure include administering multiple doses of an anti-CEACAM5 antibody, or an immunoconjugate comprising the antibody, to a patient over a specified time course. For example, the anti-CEACAM5 antibody, or an immunoconjugate comprising the antibody, can be administered about 1 to 5 times per day, about 1 to 5 times per week, about 1 to 5 times every two weeks, about 1 to 5 times per month or about 1 to 5 times per year. In certain embodiments, the methods of the disclosure include administering a first dose of anti-CEACAM5 antibody, or an immunoconjugate comprising the antibody, to a patient at a first time point, followed by administering at least a second dose of anti-CEACAM5 antibody, or an immunoconjugate comprising the antibody, to the patient at a second time point. The first and second doses, in certain embodiments, may contain the same amount of anti-CEACAM5 antibody, or of an immunoconjugate comprising the antibody. The time between the first and second doses may be from about a few hours to several weeks. For example, the second time point (i.e., the time when the second dose is administered) can be from about 1 hour to about 7 weeks after the first time point (i.e., the time when the first dose is administered). According to certain exemplary embodiments of the present disclosure, the second time point can be about 1 hour, about 4 hours, about 6 hours, about 8 hours, about 10 hours, about 12 hours, about 24 hours, about 2 days, about 3 days, about 4 days, about 5 days, about 6 days, about 7 days, about 2 weeks, about 3 weeks, about 4 weeks, about 6 weeks, about 8 weeks, about 10 weeks, about 12 weeks, about 14 weeks or longer after the first time point. In certain embodiments, the second time point is about 1 week or about 2 weeks. Third and subsequent doses may be similarly administered throughout the course of treatment of the patient. The disclosure provides methods of using therapeutic compositions comprising anti-CEACAM5 antibodies or antigen-binding fragments thereof, or immunoconjugates comprising the antibodies, and, optionally, one or more additional therapeutic agents.

According to an embodiment, the antibody or immunoconjugate is administered at a dose of 80, 100, 120, 135, 150 or 170 mg/m², as a loading dose.

Within the disclosure, the expression “loading dose” intends to refer to a dose of drug used at a start of a treatment to frontload an adequate plasma concentration of the drug that will be subsequently maintained by a maintenance dose. A loading dose is typically higher than a maintenance dose. A “maintenance dose” intends to refer to a dose of a drug, which is administered on a regular schedule, once a high plasma concentration of the drug has been established through the use of a loading dose, to maintain a plateau of the plasma drug concentration. A maintenance dose is lower than a loading dose.

According to an embodiment, the antibody or immunoconjugate is administered at a dose of 120, 135, 150 or 170 mg/m², as a loading dose.

In a particular embodiment, the antibody or immunoconjugate is administered at a dose of from about 100 mg/m² to about 200 mg/m² as a loading dose, in particular of about 135 mg/m², about 150 mg/m² or about 170 mg/m², as a loading dose.

According to an embodiment, the antibody or immunoconjugate is administered at a dose of 100 mg/m², as a maintenance dose.

According to an embodiment, the antibody or immunoconjugate is administered at a dose of 120, 135, 150 or 170 mg/m², as a loading dose, on cycle 1, and then at a dose of 100 mg/m², as a maintenance dose, on additional cycle(s).

According to an embodiment, the antibody or immunoconjugate is administered at a dose level of 150 mg/m², on cycle 1, and the cycle is about 2 weeks.

According to an embodiment, the antibody or immunoconjugate is administered at a dose of 150 mg/m², on cycle 1, as a loading dose, and at a dose of 100 mg/m², as a maintenance dose, on additional cycle(s). The cycle(s) may be about 2 weeks.

According to an embodiment, the antibody or immunoconjugate is administered at a dose of 170 mg/m², on cycle 1, and the cycle is about 2 or 3 weeks.

According to an embodiment, the antibody or immunoconjugate is administered at dose of 170 mg/m², as a loading dose, on cycle 1, and at a dose of 100 mg/m², as a maintenance dose, on additional cycle(s). The cycle(s) may be about 2 or 3 weeks.

According to an embodiment, the antibody or immunoconjugate is administered at a dose of 100 mg/m² on all cycles, i.e., on cycle 1 and on additional cycle(s). The cycle(s) may be about 2 or 3 weeks.

In accordance with the methods disclosed herein, the anti-CEACAM5 antibody, or an immunoconjugate comprising the antibody, (or pharmaceutical formulation comprising the antibody, or an immunoconjugate comprising the antibody) can be administered to the patient using any acceptable device or mechanism. For example, the administration can be accomplished using a syringe and needle or with a reusable pen and/or autoinjector delivery device. The methods of the present disclosure include the use of numerous reusable pen and/or autoinjector delivery devices to administer an anti-CEACAM5 antibody, or an immunoconjugate comprising the antibody (or pharmaceutical formulation comprising the antibody, or an immunoconjugate comprising the antibody). Examples of such devices include, but are not limited to AUTOPEN (Owen Mumford, Inc., Woodstock, UK), DISETRONIC pen (Disetronic Medical Systems, Bergdorf, Switzerland), HUMALOG MIX 75/25 pen, HUMALOG pen, HUMALIN 70/30 pen (Eli Lilly and Co., Indianapolis, IN), NOVOPEN I, II and III (Novo Nordisk, Copenhagen, Denmark), NOVOPEN JUNIOR (Novo Nordisk, Copenhagen, Denmark), BD pen (Becton Dickinson, Franklin Lakes, NJ), OPTIPEN, OPTIPEN PRO, OPTIPEN STARLET, and OPTICLIK (Sanofi-Aventis, Frankfurt, Germany). Examples of disposable pen and/or autoinjector delivery devices having applications in subcutaneous delivery of a pharmaceutical composition of the present disclosure include, but are not limited to the SOLOSTAR pen (Sanofi-Aventis), the FLEXPEN (Novo Nordisk), and the KWIKPEN (Eli Lilly), the SURECLICK Autoinjector (Amgen, Thousand Oaks, CA), the PENLET (Haselmeier, Stuttgart, Germany), the EPIPEN (Dey, L.P.), and the HUMIRA Pen (AbbVie Inc., North Chicago, IL), to name only a few.

In one embodiment, the antibody, or an immunoconjugate comprising the antibody, is administered with a prefilled syringe. In another embodiment, the antibody, or an immunoconjugate comprising the antibody, is administered with a prefilled syringe containing a safety system. For example, the safety system prevents an accidental needle-stick injury. In various embodiments, the antibody is administered with a prefilled syringe containing an ERIS safety system (West Pharmaceutical Services Inc.). See also U.S. Pat. Nos. 5,215,534 and 9,248,242, incorporated herein by reference in their entireties.

In another embodiment, the antibody, or an immunoconjugate comprising the antibody, is administered with an auto-injector. In various embodiments, the antibody, or an immunoconjugate comprising the antibody, is administered with an auto-injector featuring the PUSHCLICK technology (SHL Group). In various embodiments, the autoinjector is a device comprising a syringe that allows for administration of a dose of the composition and/or antibody to a subject. See also U.S. Pat. Nos. 9,427,531 and 9,566,395, incorporated herein by reference in their entireties.

The use of a microinfusor to deliver an anti-CEACAM5 antibody, or an immunoconjugate comprising the antibody (or pharmaceutical formulation comprising the antibody, or an immunoconjugate comprising the antibody) to a patient is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996), incorporated herein by reference in their entireties. Microinfusors are particularly useful for the delivery of large doses of therapeutic proteins contained within high concentration and/or viscous solutions.

### Combined Treatment

According to the present disclosure, the antibody-drug conjugate comprising an anti-CEACAM5-antibody is for use for treating cancer in combination with an agent or drug for treatment of said neuroendocrine cancers. The disclosure also relates to an agent or drug for treatment of a neuroendocrine cancer in combination with the antibody-drug conjugate comprising an anti-CEACAM5-antibody.

In a particular embodiment, the subject is pre-treated with an agent or drug for treatment of neuroendocrine cancers expressing CEACAM5.

The present disclosure also relates to a method of treatment of cancer in a subject in need thereof, comprising administering the antibody-drug conjugate comprising an anti-CEACAM5-antibody, and administering an agent or drug for treatment of said neuroendocrine cancer to a subject in need thereof.

The present disclosure also relates to a combination comprising an agent or drug for treatment of a neuroendocrine cancer and an antibody-drug conjugate comprising an anti-CEACAM5-antibody for use for treating said cancer.

A method of treatment or a use, as disclosed herein, may achieve an improved effect in reducing tumor size.

A method of treatment or a use, as disclosed herein, may achieve an improved effect in inhibiting tumor growth.

The present disclosure also relates to a combination for the manufacture of a medicament for the treatment of a neuroendocrine cancer, comprising an agent or drug for treatment of said neuroendocrine cancer and an antibody-drug conjugate comprising an anti-CEACAM5-antibody.

In an embodiment, said combination permits a simultaneous, separate or a sequential administration of an agent or drug for treatment of a neuroendocrine cancer and the antibody-drug conjugate comprising an anti-CEACAM5-antibody.

“Simultaneous administration” intends to refer to an administration of both agents at the same time and at the same body location.

“Sequential administration” intends to refer to an administration of both agents at the two successive times, distant each other from at least 30 minutes to several hours, days, weeks, or months, and at different or same body locations.

“Separate administration” intends to refer to an administration of both agents in a short interval of times, i.e., from a few seconds, ca. 10 to 30 seconds, to a few minutes, ca. 1 to less than 10 minutes, and at same or different body location(s).

In an embodiment, said combination permits a simultaneous administration of an agent or drug for treatment of a neuroendocrine cancer and the antibody-drug conjugate comprising an anti-CEACAM5-antibody.

In an embodiment, said combination permits a separate administration of an agent or drug for treatment of a neuroendocrine cancer and the antibody-drug conjugate comprising an anti-CEACAM5-antibody.

In an embodiment, said combination permits a sequential administration of an agent or drug for treatment of a neuroendocrine cancer and the antibody-drug conjugate comprising an anti-CEACAM5-antibody.

In a further embodiment, combinations according to the disclosure are pharmaceutical compositions or kits-of-parts.

According to some embodiments, the antibody-drug conjugate comprising an anti-CEACAM5-antibody and an agent or drug for treatment of a neuroendocrine cancer are administered simultaneously, separately, or sequentially to a subject in need thereof.

According to an embodiment, the antibody-drug conjugate comprising an anti-CEACAM5-antibody and an agent or drug for treatment of a neuroendocrine cancer are simultaneously administered to a subject in need thereof. For example, antibody-drug conjugate comprising an anti-CEACAM5-antibody and an agent or drug for treatment of a neuroendocrine cancer are administered on day one of a cycle, approximatively at the same time.

According to an embodiment, the antibody-drug conjugate comprising an anti-CEACAM5-antibody and an agent or drug for treatment of a neuroendocrine cancer are separately administered to a subject in need thereof. For example, antibody-drug conjugate comprising an anti-CEACAM5-antibody and an agent or drug for treatment of a neuroendocrine cancer are administered on day one of a cycle, by separate routes or at separates locations of the body of said subject.

According to an embodiment, the antibody-drug conjugate comprising an anti-CEACAM5-antibody and an agent or drug for treatment of a neuroendocrine cancer are sequentially administered to a subject in need thereof. For example, antibody-drug conjugate comprising an anti-CEACAM5-antibody and an agent or drug for treatment of a neuroendocrine cancer are administered on day one of a cycle, at different times.

In a further embodiment, the antibody-drug conjugate comprising an anti-CEACAM5-antibody and an agent or drug for treatment of a neuroendocrine cancer are formulated (i) in a single pharmaceutical composition comprising the antibody-drug conjugate and the agent or drug for treatment of a neuroendocrine cancer, or (ii) in the form of two separate pharmaceutical compositions, wherein one pharmaceutical composition comprises the antibody-drug conjugate comprising an anti-CEACAM5-antibody, and the other pharmaceutical composition comprises the agent or drug for treatment of a neuroendocrine cancer. The pharmaceutical composition(s) comprise(s) at least one pharmaceutically acceptable excipient.

In the case of formulation in two separate pharmaceutical compositions, the two separate pharmaceutical compositions may be administered simultaneously, separately, or sequentially, to a subject in need thereof.

In some embodiments, the pharmaceutical compositions are sequentially administered.

In a sequential administration, the period of time between the administration of the agent or drug for treatment of a neuroendocrine cancer and the antibody-drug conjugate comprising an anti-CEACAM5-antibody may last from about a few minutes to about several hours, days, or weeks. In some embodiments, the period of time may range from about 5 minutes to about 3 hours, for example from 10 minutes to about 2.5 hours, from about 30 minutes to about 2 hours, or from about 1 hour to about 1.5 hours. A period of time between may last about 5 minutes, about 10 minutes, about 30 minutes, about 1 hour, 1.5 hours, about 2 hours, about 2.5 hours or about 3 hours.

In some embodiments, in a sequential administration on a same day of a cycle, the period of time between the administration of the agent or drug for treatment of a neuroendocrine cancer and the antibody-drug conjugate comprising an anti-CEACAM5-antibody may range from about 5 minutes to about 3 hours, for example from 10 minutes to about 2.5 hours, from about 30 minutes to about 2 hours, or from about 1 hour to about 1.5 hours. In a sequential administration on a same day of a cycle, a period of time between may last about 5 minutes, about 10 minutes, about 30 minutes, about 1 hour, 1.5 hours, about 2 hours, about 2.5 hours or about 3 hours.

In a sequential administration, the antibody-drug conjugate (ADC) comprising an anti-CEACAM5-antibody may be administered after or before the agent or drug for treatment of a neuroendocrine cancer.

In some embodiments, the antibody-drug conjugate comprising an anti-CEACAM5-antibody may be administered before the agent or drug for treatment of a neuroendocrine cancer.

In some embodiments, the antibody-drug conjugate comprising an anti-CEACAM5-antibody may be administered after the agent or drug for treatment of a neuroendocrine cancer.

In some embodiments, the sequence of administration of the antibody-drug conjugate comprising an anti-CEACAM5-antibody and of the agent or drug for treatment of a neuroendocrine cancer may be the same for all cycles of treatment.

In some embodiments, the sequence of administration of the ADC and of the agent or drug for treatment of a neuroendocrine cancer may vary along the cycles of treatment. In some embodiments, one or more cycles of a treatment may comprise a first sequence of administration and one or more cycles of said may comprise a second sequence of administration, the first and second sequences being different.

In some embodiments, for example in a use for treating a cancer, in a first cycle of treatment, the antibody-drug conjugate comprising an anti-CEACAM5-antibody may be administered after the agent or drug for treatment of a neuroendocrine cancer, and in a subsequent additional cycle of treatment the antibody-drug conjugate comprising an anti-CEACAM5-antibody may be administered after the agent or drug for treatment of a neuroendocrine cancer.

In some embodiments, for example in a use for treating a cancer, in a first cycle of treatment, the antibody-drug conjugate comprising an anti-CEACAM5-antibody may be administered after the agent or drug for treatment of a neuroendocrine cancer, and in a subsequent additional cycle of treatment the antibody-drug conjugate comprising an anti-CEACAM5-antibody may be administered before the agent or drug for treatment of a neuroendocrine cancer.

In some embodiments, for example in a use for treating a cancer, in a first cycle of treatment, the antibody-drug conjugate comprising an anti-CEACAM5-antibody may be administered before the agent or drug for treatment of a neuroendocrine cancer, and in a subsequent additional cycle of treatment the antibody-drug conjugate comprising an anti-CEACAM5-antibody may be administered before the agent or drug for treatment of a neuroendocrine cancer.

In some embodiments, for example in a use for treating a cancer, in a first cycle of treatment, the antibody-drug conjugate comprising an anti-CEACAM5-antibody may be administered before the agent or drug for treatment of a neuroendocrine cancer, and in a subsequent additional cycle of treatment the antibody-drug conjugate comprising an anti-CEACAM5-antibody may be administered after the agent or drug for treatment of a neuroendocrine cancer.

In some embodiments, for example in a use for treating a cancer such as neuroendocrine small cell lung carcinoma (SCLC), the antibody-drug conjugate comprising an anti-CEACAM5-antibody may be administered after the agent or drug for treatment of a neuroendocrine cancer for all cycles of treatment.

In some embodiments, the agent or drug for treatment of a neuroendocrine cancer may be administered at a loading dose in a first cycle and at a maintenance dose in additional cycle(s).

In some embodiments, the agent or drug for treatment of a neuroendocrine cancer may be administered at a same dose in a first cycle and additional cycle(s).

In some embodiments, the antibody-drug conjugate comprising an anti-CEACAM5-antibody may be administered at a loading dose in a first cycle and at a maintenance dose in additional cycle(s), and the agent or drug for treatment of a neuroendocrine cancer may be administered at a same dose in a first cycle and additional cycle(s).

In some embodiments, the antibody-drug conjugate comprising an anti-CEACAM5-antibody may be administered at a same dose in a first cycle and additional cycle(s) and the agent or drug for treatment of a neuroendocrine cancer may be administered at a same dose in a first cycle and additional cycle(s).

In some embodiments, the antibody-drug conjugate and the agent or drug for treatment of a neuroendocrine cancer may be administered at day 1 of a first cycle of treatment and at day 1 of an at least one additional cycle(s) of treatment.

The antibody-drug conjugate and the agent or drug for treatment of a neuroendocrine cancer may be administered at day 1 of each cycle of treatment.

Said pharmaceutical compositions may be as those described above.

All publications mentioned herein are incorporated herein by reference in their entirety for all purposes.

SEQUENCE LISTING

SEQ ID NO: 1: variable domain of heavy chain of anti-CEACAM5
antibody
EVQLQESGPGLVKPGGSLSLSCAASGFVFSSYDMSWVRQTPERGLEWVAYISSGGG
ITYAPSTVKGRFTVSRDNAKNTLYLQMNSLTSEDTAVYYCAAHYFGSSGPFAYWGQGTLVT
VSS
SEQ ID NO: 2: variable domain of light chain of anti-CEACAM5
antibody
DIQMTQSPASLSASVGDRVTITCRASENIFSYLAWYQQKPGKSPKLLVYNTRTLAE
GVPSFSGSGSGTDFSLTISSLQPEDFATYYCQHHYGTPFTFGSGTKLEIK
SEQ ID NO: 3: HCDR1 of anti-CEACAM5 antibody
GFVFSSYD
SEQ ID NO: 4: HCDR2 of anti-CEACAM5 antibody
ISSGGGIT
SEQ ID NO: 5: HCDR3 of anti-CEACAM5 antibody
AAHYFGSSGPFAY
SEQ ID NO: 6: LCDR1 of anti-CEACAM5 antibody
ENIFSY
SEQ ID NO:7: LCDR3 of anti-CEACAM5 antibody
QHHYGTPFT
SEQ ID NO: 8: heavy chain of the anti-CEACAM5 antibody
EVQLQESGPGLVKPGGSLSLSCAASGFVFSSYDMSWVRQTPERGLEWVAYISSGGG
ITYAPSTVKGRFTVSRDNAKNTLYLQMNSLTSEDTAVYYCAAHYFGSSGPFAYWGQGTLVTV
SSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNVF
SCSVMHEALHNHYTQKSLSLSPG
SEQ ID NO: 9: light chain of the anti-CEACAM5 antibody
DIQMTQSPASLSASVGDRVTITCRASENIFSYLAWYQQKPGKSPKLLVYNTRTLAEGVP
SRFSGSGSGTDFSLTISSLQPEDFATYYCQHHYGTPFTFGSGTKLEIKRTVAAPSVFIFPPS
DEQLKSGTASVVCLLNNFYPREAKVQWKVDNALQSGNSQESVTEQDSKDSTYSLSSTLTLS
KADYEKHKVYACEVTHQGLSSPVTKSFNRGEC

### EXAMPLES

### Example 1: Expression of CEACAM5 in Neuroendocrine Carcinomas

### Study Group

The total study group was made of 182 patients, with 183 samples available (one patient had two different samples available at different time points of his course). Inclusion criteria were:
Diagnosis of neuroendocrine carcinoma, small cell or large cell type, based on morphological and immunohistochemical features (since there is no consensus on the minimal criteria required to diagnose as neuroendocrine a poorly differentiated carcinoma, the inventors retained all tumors showing expression of at least one neuroendocrine marker in a significant proportion of tumor cells with suggestive morphology)
No evidence of mixed tumor, defined by the presence of a non-neuroendocrine component associated with the neuroendocrine component
Tissue material of sufficient quantity, quality and representativity for histological study and immunohistochemical evaluation

### Material and Methods

The following data were recorded: site of sample studied, date of sample studied, primary or metastatic nature of the sample studied, histological subtype.

Most of the samples available for histological and immunohistochemical study were biopsy specimens; a few surgical specimens were available (it is reminded that neuroendocrine carcinomas are rarely accessible to surgery). A histological review by an expert pathologist of all tissue material available was performed to assess the quantity, quality and representativity of tissue samples and to verify the initial diagnosis.

Immunohistochemical detection of CEACAM5 was performed as of current protocols (technique and interpretation). The detection was performed on an automated stainer (Ventana Benchmark Ultra, Ventana, Tucson, AZ). 4 μm-thick sections of formalin-fixed paraffin-embedded tissue were deparaffinized according to standard procedures. They were incubated 20 min in unmasking solution CC1 (Ventana), pH8, at 95° C., then with the primary antibody (provided by Sanofi), diluted 1/3760 (final concentration 2.39 μg·mL-1). Revelation was performed with ultraView DAB universal detection kit (Ventana). A light nuclear counterstaining was performed with hematoxylin. All samples were examined by the same pathologist. All tumor cells with either membranous or cytoplasmic staining were counted positive, since, especially in the small cell type, the exact location of labelling is difficult to ascertain in such tumor cells, with high nucleo-cytoplasmic ratio and poorly abundant cytoplasm.

A scoring system was used: 0, no positive tumor cell detected; 1, <5% positive tumor cells; 2, 5-50% positive tumor cells; 3, 50-80% positive tumor cells; 4, >80% positive tumor cells.

### Results

### Characteristics of the Study Group

183 samples from 182 patients were included. 133 were from primary sites, 50 from metastatic sites, including liver (n=20), lymph nodes (n=16), peritoneum (n=6), bone (n=3), skin (n=2), lung (n=1).

The distribution of cases according to the location of the primary is given in Table 1.

TABLE 1

| | | Total | Primary | Metastatic |
|---|---|---|---|---|
| Location of the primary | | number | site | site |
| Digestive tract | Esophagus | 14 | 13 | 1 |
| | Stomach | 10 | 6 | 4 |
| | Duodenum | 1 | 1 | 0 |
| | Colon | 14 | 10 | 4 |
| | Rectum | 9 | 6 | 3 |
| | Anal region | 1 | 0 | 1 |
| Pancreas | | 23 | 17 | 6 |
| Breast | | 3 | 2 | 1 |
| Female organs | Ovaries | 3 | 2 | 1 |
| | Endometrium | 8 | 8 | 0 |
| | Uterine cervix | 15 | 13 | 2 |
| Head and neck | | 26 | 20 | 6 |
| Skin | Merkel cell | 31 | 25 | 6 |
| | carcinoma | | | |
| Urinary tract | Bladder | 9 | 0 | 9 |
| Thymus | | 1 | 1 | 0 |
| Others | Presacral | 1 | 1 | 0 |
| | Retroperitoneum | 1 | 1 | 0 |
| Unknown* | | 13 | 7 | 6 |

*patients presenting with disseminated disease, without predominant location

111 cases were classified as small cell type, 41 as large cell type (mainly from stomach, colon, pancreas, endometrium, head and neck). 31 cases were from a distinctive subset, termed Merkel cell carcinoma, restricted to the skin.

### Expression of CEACAM5

Tumor cell expression of CEACAM5 was detected in 53 samples (29%); the score was 3 or 4 in 20 samples (11%). Interestingly, for the patient with two different samples, scoring was 0 in one and 1 in the other.

In primary sites, tumor cell expression of CEACAM5 was detected in 35 samples (26.3%); the score was 3 or 4 in 12 samples (9%).

In metastatic sites, tumor cell expression of CEACAM5 was detected in 18 samples (36%); the score was 3 or 4 in 8 samples (16%).

The expression profile according to the location of the primary, including both primary and metastatic sites, is given in Table 2.

TABLE 2

| | | Total | Positive | Scores 3-4 |
|---|---|---|---|---|
| Location of the primary | | number | (n, (%)) | (n, (%)) |
| Digestive tract | Esophagus | 14 | 4 (29%) | 2 (14.2%) |
| | Stomach | 10 | 2 (20%) | 1 (10%) |
| | Duodenum | 1 | 0 | 0 |
| | Colon | 14 | 3 (21.5%) | 1 (7%) |
| | Rectum | 9 | 2 (22.2%) | 1 (11.1%) |
| | Anal region | 1 | 1 | 0 |
| Pancreas | | 23 | 5 (21.8%) | 3 (13%) |
| Breast | | 3 | 0 | 0 |
| Female organs | Ovaries | 3 | 0 | 0 |
| | Endometrium | 8 | 3 (37.5%) | 0 |
| | Uterine cervix | 15 | 9 (60%) | 2 (13.3%) |
| Head and neck | | 26 | 13 (50%) | 7 (19.2%) |
| Skin | Merkel cell | 31 | 0 | 0 |
| | carcinoma | | | |
| Urinary tract | Bladder | 9 | 5 (55.5%) | 1 (11.1%) |
| Thymus | | 1 | 0 | 0 |
| Others | Presacral | 1 | 0 | 0 |
| | Retroperitoneum | 1 | 0 | 0 |
| Unknown | | 13 | 6 (46.2%) | 4 (30.8%) |

The expression profiles according to the location of the primary, for, respectively, the primary and metastatic sites, are given in Tables 3 and 4.

TABLE 3
(Primary sites)

| | | Total | Positive | Scores 3-4 |
|---|---|---|---|---|
| Location of the primary | | number | (n, (%)) | (n, (%)) |
| Digestive tract | Esophagus | 13 | 4 (30.7%) | 2 (15.4%) |
| | Stomach | 6 | 0 | 0 |
| | Colon | 10 | 3 (30%) | 1 (10%) |
| | Rectum | 6 | 1 (16.7%) | 0 |
| | Anal region | 0 | 0 | 0 |
| Pancreas | | 17 | 3 (17.6%) | 2 (11.8%) |
| Female organs | Endometrium | 8 | 3 (37.5%) | 0 |
| | Uterine cervix | 13 | 8 (61.5%) | 2 (15.4%) |
| Head and neck | | 20 | 11 (55%) | 4 (20%) |
| Urinary tract | Bladder | 0 | 0 | 0 |
| Unknown* | | 7 | 2 (28.6%) | 1 (14.3%) |

*the site sampled is known to be potentially involved by primary neuroendocrine carcinoma.

TABLE 4
(Metastatic sites)

| | | Total | Positive | Scores 3-4 |
|---|---|---|---|---|
| Location of the primary | | number | (n, (%)) | (n, (%)) |
| Digestive tract | Esophagus | 1 | 0 | 0 |
| | Stomach | 4 | 2 (50%) | 1 (25%) |
| | Colon | 4 | 0 | 0 |
| | Rectum | 3 | 1 | 1 |
| | Anal region | 1 | 1 | 0 |
| Pancreas | | 6 | 2 (33.3%) | 1 (16.7%) |
| Female organs | Endometrium | 0 | 0 | 0 |
| | Uterine cervix | 2 | 1 | 0 |
| Head and neck | | 6 | 2 (33.3%) | 1 (16.7%) |
| Urinary tract | Bladder | 9 | 5 (55.5%) | 1 (11.1%) |
| Unknown* | | 6 | 4 (66.7%) | 3 (50%) |

*the site sampled is not known to be potentially involved by primary neuroendocrine carcinoma (e.g., liver, lymph node or bone).

### Characteristics of CEACAM5+ Neuroendocrine Carcinomas

There was no significant difference in the number of positive samples between small cell and large cell neuroendocrine carcinomas. Among the 20 samples with scores 3-4, 16 were from small cell neuroendocrine carcinoma (14.5%) and 4 from large cell neuroendocrine carcinoma (10%).

### Preliminary Conclusions

This series of 183 samples is fairly representative of the various locations in which neuroendocrine carcinomas may occur, including some rare and less studied sites (female organs, head and neck). Some sites are not represented, either because they have been voluntarily excluded (lung, prostate), or because they are not (or exceptionally) involved, for unknown reasons, by neuroendocrine carcinoma (liver, small intestine, appendix). There is some bias in the composition of the series due to the specificities of the recruitment of Gustave Roussy: for instance, the absence of urological surgery in the institution explains why only metastatic sites were available for the urinary bladder.

About 30% of samples contained at least a few positive cells. The example of the patient with two different samples available (one sample with no positive cell found, the other one with <5% positive cells) illustrates the fact that the identification of only a few positive cells is unpredictable and depends on the extent of material studied. Scores 3-4 (50-100% positive cells) are likely to be more reliable: they were observed in 11% of samples.

Four locations were enriched in positive cases:
endometrium: 37.5% of positive samples, but none score 3-4
uterine cervix: 60% of positive samples, 13.3% score 3-4
head and neck: 50% of positive samples, 19.2% score 3-4
urinary bladder: 55.5% of positive samples, 11% score 3-4.

These results are original and interesting, since all four locations are among the rare subsets of neuroendocrine carcinoma, consequently the less studied and those with the most unmet therapeutic needs.

Neuroendocrine carcinomas of unknown primary are a frequent clinical problem. The term is used either for patients presenting with a disseminated disease without any predominant location or, more rarely, for patients diagnosed in a site not known to be involved by primary neuroendocrine carcinoma (such as the liver or lymph nodes), without evidence of any other tumor mass. In this study, this category is among the most enriched in positive cases: 46.2% of positive samples, 31% score 3-4. This might be of clinical relevance.

Further, the percentage of positive samples was slightly higher in metastatic than in primary sites, but the difference was not statistically significant (chi-2 test, p>0.15). It is important to reassess that neuroendocrine carcinoma is usually diagnosed at an advanced stage and that the therapeutic challenge is to propose a second line treatment for a systemic disease, not a specific treatment targeting late-onset metastases. The possible difference observed between primary and metastatic sites, if confirmed by further studies, would therefore not be clinically relevant.

In conclusion, this large series shows that a significant number of cases of neuroendocrine carcinoma, irrespectively of the site of the primary, express CEACAM5+ in the majority of tumor cells (about 10% in the whole series); the number of such positive cases is even higher in some particular locations (including head and neck, female organs, urinary bladder). The screening can be done by immunohistochemistry, since all patients have at least one diagnostic biopsy and that re-biopsy is not an issue here, because of the disseminated nature of the disease at presentation and its rapid course.

### Example 2: Expression of CEACAM5 in Medullary Thyroid Carcinoma

### Objective

To assess CEACAM5 expression in medullary thyroid carcinoma.

### Study Samples

Number of samples: 40
Origin: surgical resection (thyroidectomy)
Stages distribution (known for 31/40 samples): 19% stage 1, 36% stage 2, 19% stage 3, 26% stage 4

### Material & Methods

Immunohistochemistry (IHC) using the murine parental antibody (769-cea-4) of antibody Tusamitamab ravtansine was performed to assess the level and subcellular localization of CEACAM5 protein in the studied samples. IHC was carried out using the Ventana Discovery XT automated system with company recommended reagents (Ventana Medical Systems Inc, USA). Formalin-fixed paraffin-embedded human tissue slides were dewaxed and pretreated with cell-conditioning buffer at 95° C. for 48 minutes. Slides were incubated with 769-cea-4 or isotype control mouse IgG1 antibodies at 5 μg/mL final concentration for 3 hours at 24° C. A rabbit anti-mouse IgG used as a specie linker antibody was incubated at 24° C. for 32 minutes at 1/200 final dilution. The ready-to-use biotin free peroxidase multimer anti-rabbit UltraMap™ was applied at 24° C. for 16 minutes. Staining was evaluated under light microscopy included histologic site, main type of reactive cell, staining intensity, and cell staining frequency.

### Results

High expression of membrane CEACAM5 was found in medullary thyroid carcinoma with median intensity and frequency of 2+(60%) and mean All red score 67%.

Global prevalence is 80% (32/40); 35% of samples expressed CEACAM5 with intensity ≥2+ and tumor cell frequency ≥50%.

### Example 3: Expression of CEACAM5 in Neuroendocrine Carcinomas of the Prostate on Patients

In this study, the inventors decided to combine morphological analysis and expression of a general neuroendocrine marker, synaptophysin, to evaluate the neuroendocrine differentiation of prostate adenocarcinoma.

### Study Group

The total study group was made of 54 patients. Inclusion criteria were:
Patients with a known history of prostate adenocarcinoma diagnosed and/or treated at Gustave Roussy Cancer Campus, including the patients participating to Moscato and MatchR trials,
Pathology report describing a morphology suggestive of either “undifferentiated”, “poorly differentiated” or “neuroendocrine” carcinoma, with or without immunohistochemical evidence of neuroendocrine differentiation,
Tissue material of sufficient quantity, quality and representativity for histological study and immunohistochemical evaluation.

### Material and Methods

The following data were recorded from clinical and pathological files when available: date of initial diagnosis of prostate adenocarcinoma, age at initial diagnosis, Gleason grade at initial diagnosis, TNM stage at initial diagnosis, date of diagnosis of neuroendocrine differentiation, site of sample studied, date of sample studied, treatments received, date of last information, status at last information.

All the samples available for histological and immunohistochemical study were biopsy specimens; no surgical specimen was available. A histological review of all tissue material available was performed to assess the quantity, quality and representativity of tissue samples and to verify the initial diagnosis.

Additional immunohistochemistry was performed. To demonstrate the neuroendocrine differentiation of prostate adenocarcinoma, synaptophysin was performed in all cases, according to standard procedures of the laboratory. The detection was performed on an automated stainer (Ventana Benchmark Ultra, Ventana, Tucson, AZ). 4 μm-thick sections of formalin-fixed paraffin-embedded tissue were deparaffinized according to standard procedures. They were incubated with the primary antibody (clone DAK-SYNAP, Dako-Agilent, Santa Clara, CA; ready-to-use). Revelation was performed with ultraView DAB universal detection kit (Ventana). A light nuclear counterstaining was performed with hematoxylin. Other specific neuroendocrine markers (such as chromogranin A, chromogranin B or INSM1) and CD56 were not performed in all cases at this stage of the study in order to preserve the available, often limited, tissue material; their expression was recorded when the corresponding tests had been performed as part of the initial diagnostic procedure or for other ancillary studies. The expression of TTF1, often expressed in neuroendocrine carcinomas irrespectively of their origin, has been recorded when performed as part of the initial diagnostic procedure.

Immunohistochemical detection of CEACAM5 was performed as of current protocols (technique and interpretation). The detection was performed on an automated stainer (Ventana Benchmark Ultra, Ventana, Tucson, AZ). 4 μm-thick sections of formalin-fixed paraffin-embedded tissue were deparaffinized according to standard procedures. They were incubated 20 min in unmasking solution CC1 (Ventana), pH8, at 95° C., then with the primary antibody (provided by Sanofi), diluted 1/3760 (final concentration 2.39 μg·mL-1). Revelation was performed with ultraView DAB universal detection kit (Ventana). A light nuclear counterstaining was performed with hematoxylin. All samples were examined by the same pathologist. A multi-head microscope session was performed with several inventors to evaluate the results and reach a consensus in all cases.

### Results

### Clinical and Pathological Features of the Study Group

54 patients were included. Their age at initial diagnosis was usually comprised between 50 and 70 years, except from one patient with germline BRCA2 mutation, in whom the diagnosis was made at 34 years. At initial diagnosis, Gleason grade, when available, was: G6 (n=6), G7 (n=10), G8 (n=6), G9 (n=5), G10 (n=1).

33 patients presented immunohistochemical evidence of at least focal neuroendocrine differentiation. The diagnosis of neuroendocrine differentiation was made at diagnosis in 1 case or during the course of disease in 21 cases (after a delay ranging from 1 to 17 years). All cases expressed synaptophysin: 21 in >50% of tumor cells, 5 in 10-50%, 7 in <10%. Among the 21 cases with >50% synaptophysin+ tumor cells, 14 were tested for chromogranin A (9+, 5−), 2 for chromogranin B (2+) and 12 for CD56 (10+, 2−). Among the 12 remaining cases, only 4 were tested for at least one other neuroendocrine marker: all were negative.

21 patients presented no immunohistochemical evidence of neuroendocrine differentiation.

The site of the biopsy sample used in the study was:
from the primary site or from a locoregional extension in 11 cases: prostate (n=5), rectum (n=3), bladder (n=1), pelvis (n=2),
from a metastatic site in 43 cases: liver (n=20), lymph node (18), adrenal (n=1), bone (n=3), peritoneum (n=1).

### Expression of CEACAM5

Membrane expression of CEACAM5 was detected in ≥1% of tumor cells in 8 cases (range: 1-80%). In 2 additional cases, membrane expression was observed in a few scattered tumor cells (<1%). Details of the expression pattern (membrane or cytoplasm location, apparent intensity) are given in each case in the table 5 below.

TABLE 5

| | | % | | % | | | |
|---|---|---|---|---|---|---|---|
| | | positive | Intensity | positive | Intensity | % positive | |
| | | cells at | of | cells in | of | tumor cells | |
| Histological | CEACAM | the | membrane | the | cytoplasmic | for | |
| morphology | expression | membrane | staining | cytoplasm | staining | synaptophysine | Biopsy site |
| Neuroendocrine | Positive/negative | 0 | 0 | 5 | 2 | 50-100% | LIVER |
| Neuroendocrine | Positive/negative | 0 | 0 | 30 | 2 | 50-100% | LYMPH |
| | | | | | | | NODE |
| | | | | | | | REGIONAL |
| Neuroendocrine | Positive | 1 | 3 | 0 | 0 | 50-100% | LIVER |
| Neuroendocrine | Positive | <1 | 2 | 0 | 0 | 50-100% | LIVER |
| Neuroendocrine | Positive | <1 | 2 | 0 | 0 | 50-100% | LIVER |
| Neuroendocrine | Positive | 5 | 1 | 0 | 0 | 50-100% | LIVER |
| Neuroendocrine | Positive | 10 | 2 | 80 | 2 | 50-100% | LIVER |
| Neuroendocrine | Positive | 80 | 2 | 100 | 3 | 50-100% | LIVER |
| Neuroendocrine | Positive | 20 | 3 | 0 | 0 | 50-100% | LIVER |
| Neuroendocrine | Positive | 1 | 2 | 10 | 3 | 50-100% | LYMPH |
| | | | | | | | NODE |
| | | | | | | | REGIONAL |
| Neuroendocrine | Positive | 80 | 2 | 0 | 0 | 50-100% | LYMPH |
| | | | | | | | NODE |
| | | | | | | | REGIONAL |
| Neuroendocrine | Negative | 0 | 0 | 0 | 0 | 50-100% | ADRENAL |
| Neuroendocrine | Negative | 0 | 0 | 0 | 0 | 50-100% | LIVER |
| Neuroendocrine | Negative | 0 | 0 | 0 | 0 | 50-100% | LIVER |
| Neuroendocrine | Negative | 0 | 0 | 0 | 0 | 50-100% | LIVER |
| Neuroendocrine | Negative | 0 | 0 | 0 | 0 | 50-100% | LIVER |
| Neuroendocrine | Negative | 0 | 0 | 0 | 0 | 50-100% | LIVER |
| Neuroendocrine | Negative | 0 | 0 | 0 | 0 | 50-100% | PROSTATE |
| Neuroendocrine | Negative | 0 | 0 | 0 | 0 | 50-100% | PROSTATE |
| Neuroendocrine | Negative | 0 | 0 | 0 | 0 | 50-100% | LYMPH |
| | | | | | | | NODE |
| | | | | | | | REGIONAL |
| Neuroendocrine | Negative | 0 | 0 | 0 | 0 | 10-50% | RECTUM* |
| Neuroendocrine | Negative | 0 | 0 | 0 | 0 | 10-50% | PERITONEUM |
| Neuroendocrine | Negative | 0 | 0 | 0 | 0 | 10-50% | RECTUM* |
| Neuroendocrine | Negative | 0 | 0 | 0 | 0 | 10-50% | LIVER |
| Neuroendocrine | Negative | 0 | 0 | 0 | 0 | 10-50% | LIVER |
| Neuroendocrine | Negative | 0 | 0 | 0 | 0 | 0-10% | RECTUM* |
| Neuroendocrine | Negative | 0 | 0 | 0 | 0 | 0-10% | LIVER |
| Neuroendocrine | Negative | 0 | 0 | 0 | 0 | 0-10% | BONE |
| Neuroendocrine | Negative | 0 | 0 | 0 | 0 | 0-10% | LYMPH |
| | | | | | | | NODE |
| | | | | | | | DISTANT |
| Neuroendocrine | Negative | 0 | 0 | 0 | 0 | 0-10% | LYMPH |
| | | | | | | | NODE |
| | | | | | | | DISTANT |
| Neuroendocrine | Negative | 0 | 0 | 0 | 0 | 0-10% | LYMPH |
| | | | | | | | NODE |
| | | | | | | | REGIONAL |
| Neuroendocrine | Negative | 0 | 0 | 0 | 0 | 0-10% | LYMPH |
| | | | | | | | NODE |
| | | | | | | | DISTANT |
| Poorly | Negative | 0 | 0 | 0 | 0 | NEG | BLADDER* |
| differentiated | | | | | | | |
| adenocarcinoma | | | | | | | |
| Poorly | Negative | 0 | 0 | 0 | 0 | NEG | LIVER |
| differentiated | | | | | | | |
| adenocarcinoma | | | | | | | |
| Poorly | Negative | 0 | 0 | 0 | 0 | NEG | LIVER |
| differentiated | | | | | | | |
| adenocarcinoma | | | | | | | |
| Poorly | Negative | 0 | 0 | 0 | 0 | NEG | LIVER |
| differentiated | | | | | | | |
| adenocarcinoma | | | | | | | |
| Poorly | Negative | 0 | 0 | 0 | 0 | NEG | PROSTATE |
| differentiated | | | | | | | |
| adenocarcinoma | | | | | | | |
| Poorly | Negative | 0 | 0 | 0 | 0 | NEG | PROSTATE |
| differentiated | | | | | | | |
| adenocarcinoma | | | | | | | |
| Poorly | Negative | 0 | 0 | 0 | 0 | NEG | PROSTATE |
| differentiated | | | | | | | |
| adenocarcinoma | | | | | | | |
| Poorly | Negative | 0 | 0 | 0 | 0 | NEG | BONE |
| differentiated | | | | | | | |
| adenocarcinoma | | | | | | | |
| Poorly | Negative | 0 | 0 | 0 | 0 | NEG | BONE |
| differentiated | | | | | | | |
| adenocarcinoma | | | | | | | |
| Poorly | Negative | 0 | 0 | 0 | 0 | NEG | LYMPH |
| differentiated | | | | | | | NODE |
| adenocarcinoma | | | | | | | REGIONAL |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | LYMPH |
| differentiated | | | | | | | NODE |
| adenocarcinoma | | | | | | | REGIONAL |
| Poorly | Negative | 0 | 0 | 0 | 0 | NEG | LYMPH |
| differentiated | | | | | | | NODE |
| adenocarcinoma | | | | | | | REGIONAL |
| Poorly | Negative | 0 | 0 | 0 | 0 | NEG | LYMPH |
| differentiated | | | | | | | NODE |
| adenocarcinoma | | | | | | | DISTANT |
| Poorly | Negative | 0 | 0 | 0 | 0 | NEG | PELVIS* |
| differentiated | | | | | | | |
| adenocarcinoma | | | | | | | |
| Poorly | Negative | 0 | 0 | 0 | 0 | NEG | PELVIS* |
| differentiated | | | | | | | |
| adenocarcinoma | | | | | | | |

*local extension

All 8 CEACAM5+ cases expressed synaptophysin in >50% tumor cells (among a total of 21 cases with this phenotype). All positive samples were from metastatic sites: liver in 6 cases (out of 14 in this group) and lymph nodes in 2 (out of 4 in this group).

There was no detectable expression of CEACAM5 in the remaining 12 cases with synaptophysin expressed in <50% of tumor cells. There was no expression in the 21 cases without evidence of synaptophysin expression.

### Preliminary Conclusions

The study included 54 cases of prostate adenocarcinomas with undifferentiated or poorly differentiated morphology, among which 33 presented immunohistochemical evidence of at least focal neuroendocrine differentiation, as assessed by synaptophysin expression. CEACAM5 was detectable only in cases with synaptophysin expressed in >50% tumor cells. In this group of patients, CEACAM5 was detected in ≥1% of tumor cells in 8 out of 21 cases (38%). The results therefore suggest that CEACAM5 expression in prostate carcinoma is associated with the full expression of the neuroendocrine differentiation program, since synaptophysin is a terminal product of this program.

### Example 4: Antitumor Activity of Tusamitamab Ravtansine in PDX Models of Neuroendocrine Prostate Cancer

### Materials and Methods

Patient derived xenografts (PDX) of neuroendocrine pancreatic cancer were selected based on the analysis of their immunohistochemistry (IHC) and CEACAM5 expressions and grafted in mice models. Patient derived xenografts (PDX) of neuroendocrine prostate cancer tumors were selected based on both the analysis of their immunohistochemical profiling (androgen receptor (AR), Synaptophysin (SYP) and CEACAM5) and CEACAM5 mRNA expression (RNAsequencing)).

Five tumours harboring the three phenotypes (2 NEPC CEACAM5+, 1 NEPC CEACAM5focal, 1 NEPC CEACAM5- and 1 adenocarcinoma) were subsequently grafted in the flank of 30 NGS mice (NOD.CB17-Prkdcscid/NCrCrl, Charles River). Tumor growth was monitored twice a week using a digital caliper. When tumor volume reached an average of 80-200 mm3, mice were randomized and allocated to 3 different groups: Vehicle group (not injected, n=8), Irrelevant DM4-ADC (35 mg/kg, n=8) and Tusamitamab ravtansine (5 mg/kg, n=8). Irrelevant ADC or Tusamitamab ravtansine were injected intravenously in the tail of mice. Tumor growth in response to treatment was monitored twice a week and tumor volumes were calculated according to the following formula: L×W×H (L=length, W=width and H=height). Tumor growth delay was calculated as ΔT/ΔV=(median tumor size TDayY−median tumor size TDayX)/(median tumor size VDayY−median tumor size VDayX)×100 (where T=Treated and V=Vehicle and Day Y=day of measurement and DayX=day of initiation of the treatment). Mice were weighed twice a week to monitor toxicity. If 10% of the initial weight was lost mice were euthanized.

When the mean of the tumor size in the vehicle group reached 1500 mm³, mice were euthanized. Mouse tumors and plasmas were collected at euthanasia for the evaluation of circulating CEACAM5.

The expression of CEACAM5 by IHC in the different mouse models is provided in table 6 below:

TABLE 6

| SYP | | CEACAM5 | phenotype |
|---|---|---|---|
| MR-0009-P0 | +HG | − | NE |
| MR-0009-P4 | +HG | − | NE |
| MR-0041-P0 | − | − | ADK |
| MR-0041-P4 | − | − | ADK |
| MR-0077-P0 | +HG | − | NE |
| MR-0077-P4 | + | − | NE |
| MR-0084-P0 | +++ | FOCAL | NE |
| MR-0084-P4 | +++ | FOCAL | NE |
| MR-0123-P0 | − | | ADK |
| MR-0123-P4 | − | − | ADK |
| MR-0170-P0 | +HG | − | NE |
| MR-0170-P4 | +HG | − | NE |
| MR-0191-P0 | +++ | +++ | NE |
| MR-0191-P4 | ++ | ++ | NE |

wherein NE means neuroendocrine, ADK means adenocarcinoma and HG means high grade and + represents the expression grade of the considered marker (the more +, the higher the expression).

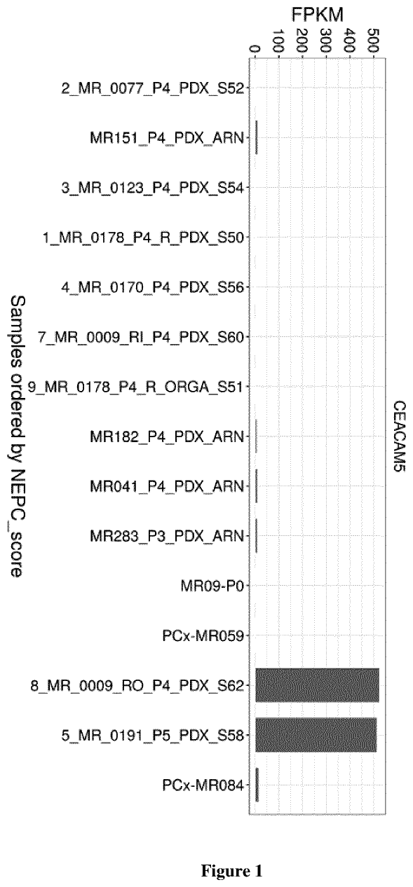

The expression of CEACAM5 measured by RNA sequencing is provided in FIG. 1.

To obtain these results, mRNA was extracted from organoids and PDXs using phenol chloroform method (Sigma, TRI reagent ref 93289). The RNA integrity (RNA Integrity Score≥7.0) was checked on the Agilent 2100 Bioanalyzer (Agilent) and quantity was determined using Qubit (Invitrogen). SureSelect Automated Strand Specific RNA Library Preparation Kit was used according to manufacturer's instructions with the Bravo Platform. Briefly, 50 to 200 ng of total RNA sample was used for poly-A mRNA selection using oligo(dT) beads and subjected to thermal mRNA fragmentation. The fragmented mRNA samples were subjected to cDNA synthesis and were further converted into double stranded DNA using the reagents supplied in the kit, and the resulting dsDNA was used for library preparation. The final libraries were bar-coded, purified, pooled together in equal concentrations and subjected to paired-end sequencing on Novaseq-6000 sequencer (Illumina) at Gustave Roussy. Sequence alignment and quantification of gene expression are processed as following. Raw reads were aligned to the human hg38/GRCh38.p7 and the mouse mm10 genomes using STAR. Reads were classified depending on their species of origin (graft or host) with XenofilteR tool. STAR was used to obtain the number of reads associated to each gene in the Gencode v26 annotation (restricted to protein-coding genes, antisense and lincRNAs). Raw counts for each sample were imported into R statistical software. Extracted count matrix was normalized for library size and coding length of genes to compute FPKM (Fragments per kilo base of transcript per million mapped fragments) expression levels.

### Results

### Tumor Shrinkage

The non-relevant ADC used at a high dose served as a positive control of the efficacy of the chemotherapeutic agent. Indeed, tumor volume significantly decreased in all models, including those with no or low expression of CEACAM5.

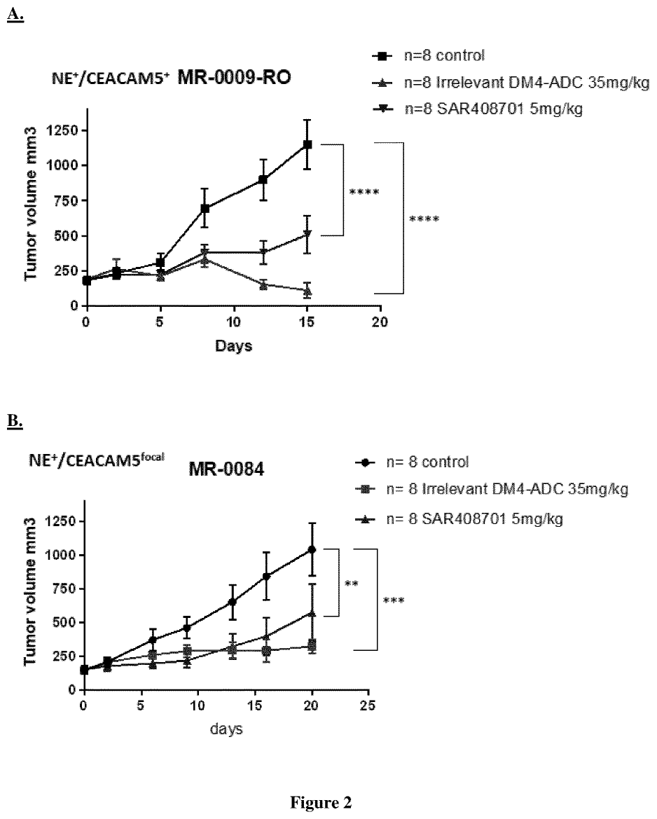

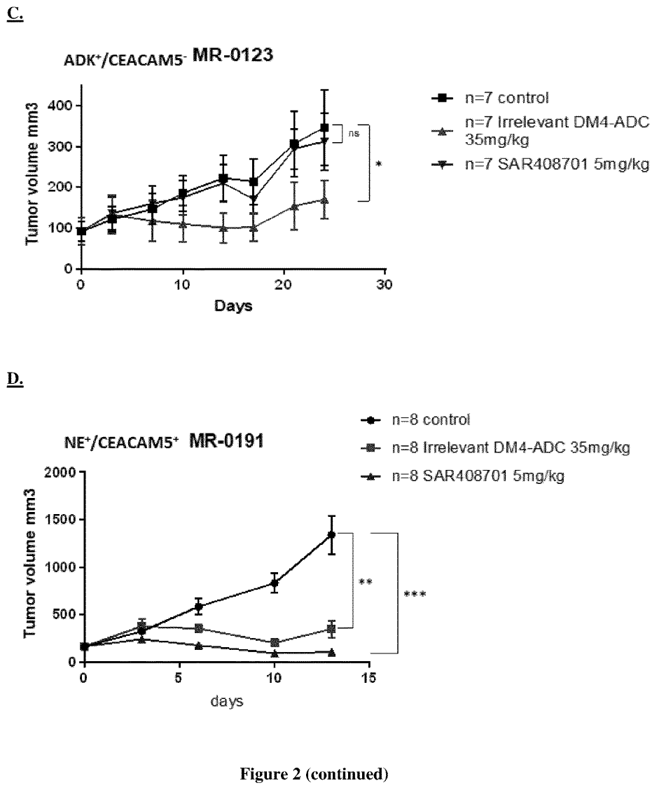

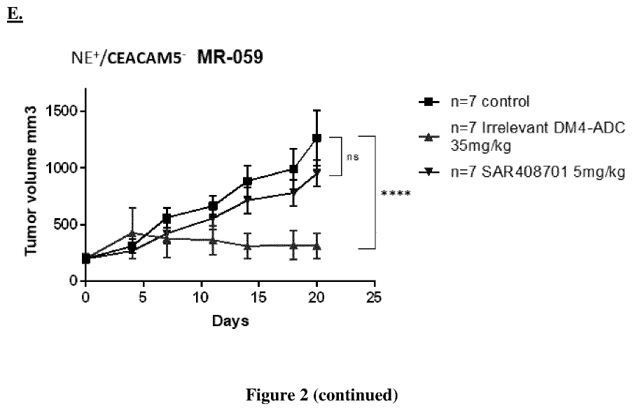

MR0191 and MR009RO (with a high expression of CEACAM5) had a significant response to a single injection of Tusamitamab ravtansine (or SAR408701) compared to the vehicle group at 21 days. At day 12, MR009RO tumor rebounded. This suggests that a second injection at day 12 could improve the response to Tusamitamab ravtansine in MR009RO (FIGS. 2A and 2D).

MR0084 classified as a CEACAM5 focal showed a significant response, to a lower extent than observed in MR191 and MR009RO (FIG. 2B).

CEACAM5-negative MR059 model and Adenocarcinoma model MR0123 showed no response to Tusamitamab ravtansine (FIGS. 2C and 2E).

### Toxicity

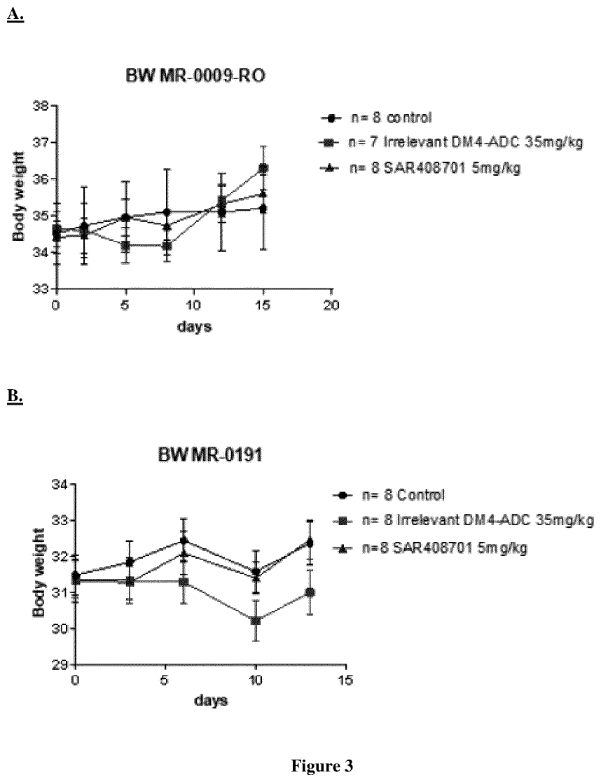

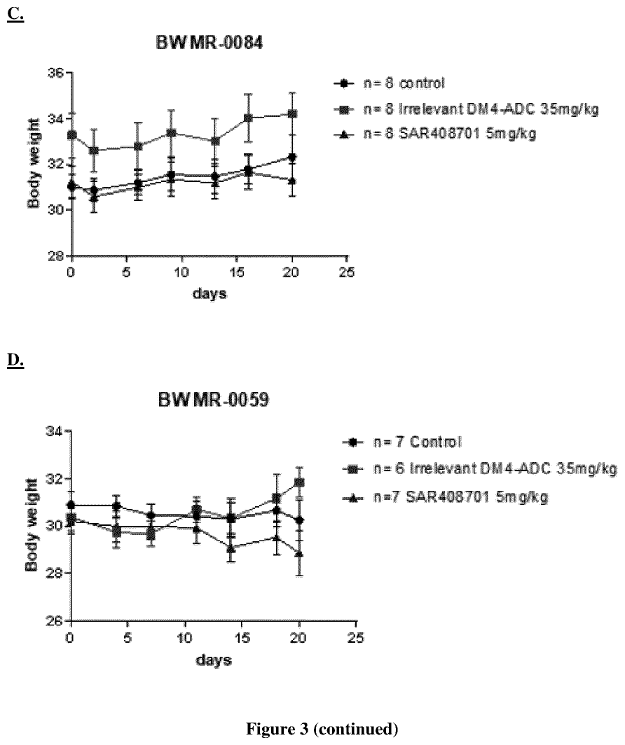

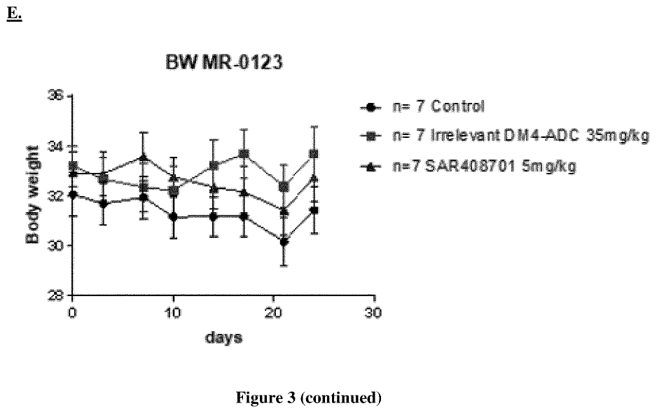

The fact that body weight evolved similarly for vehicle and Tusamitamab ravtansine treated mice show that no significant toxicity is was observed due to treatment (FIG. 3A to 3E). There was not significant difference between treated and control mice for body weight. Treated mice had a similar weight to control mice and harboured a much lower tumor burden especially in the CEACAM5+ models showing that the tumor weight is negligible compared to the mice weight.

### CEACAM 5 Expression and NEPC Markers Expression are Correlated

Reads were classified depending on their species of origin (graft or host) with XenofilteR tool. STAR was used to obtain the number of reads associated to each gene in the Gencode v26 annotation (restricted to protein-coding genes, antisense and lincRNAs). Raw counts for each sample were imported into R statistical software. Extracted count matrix was normalized for library size and coding length of genes to compute FPKM (Fragments per kilo base of transcript per million mapped fragments) expression levels.

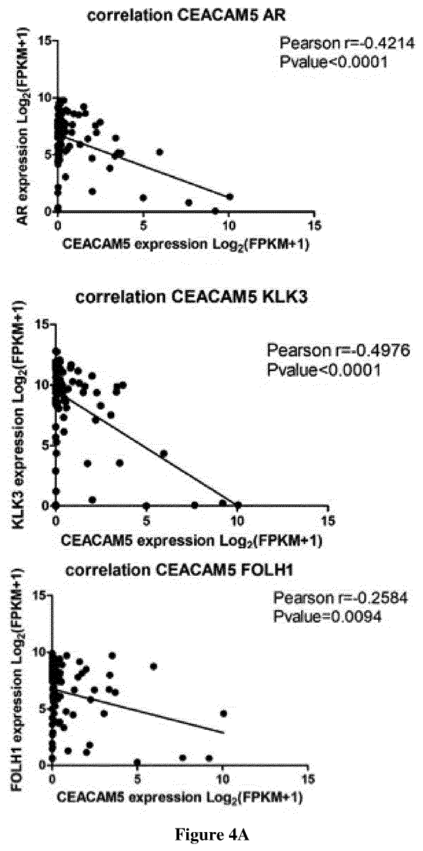

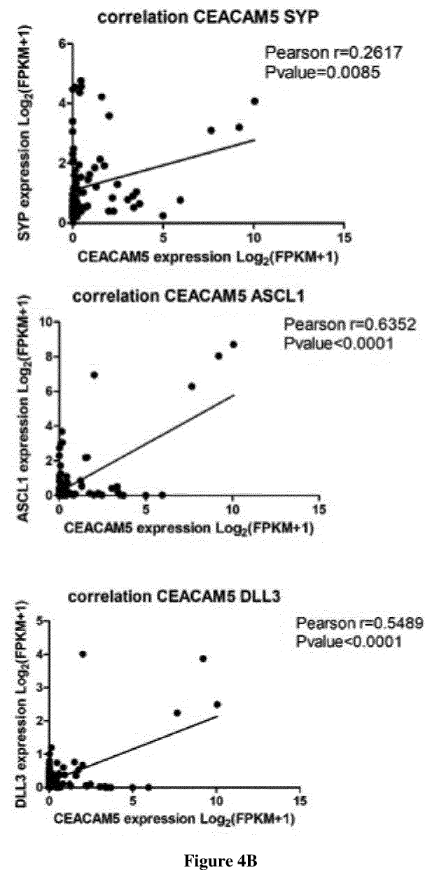

There was a significant positive correlation between CEACAM5 expression (measured by FPKM from RNA seq) and NEPC markers (measured by FPKM from RNA seq) such as SYP, ASCL1 and DLL3 and a significant negative correlation between CEACAM5 expression and luminal markers such as AR, KLK3 and FOLH1 (FIG. 4).

### Example 5: Antitumor Activity of Tusamitamab Ravtansine in Patients with Neuroendocrine Small Cell Lung Carcinoma (SCLC) (Phase I Clinical Trial)

### Primary Objective

The efficacy was assessed according to RECIST v1.1 criteria (Expansion Phase) when SAR408701 is administered once Q2W with or without a loading dose at Cycle 1 (Expansion Phase). The primary efficacy endpoint was a binary response (overall objective response), which was defined as a confirmed complete response (CR) or partial response (PR), evaluated every 4 cycles using RECIST v1.1.

### Statistical Design

This was an open label, non-randomized study with 1 cohort in SCLC to assess the efficacy of Tusamitamab ravtansine (SAR408701), administered at the recommended dose 100 mg/m2 Q2W cycle, in patients with SCLC, to establish preliminary proof-of-concept of antitumor activity in these SCLC indications.

The response rate induced under SAR408701 treatment in this trial was compared to that of the historical data with standard of care. Based on historical data in advanced stage disease, the response rate for the standard of care was assessed as 10% in the SCLC cohort; and it was expected that if SAR408701 would induce a response rate of 25%, respectively, SCLC cohorts, this would constitute outstanding antitumor activity. Using this hypothesis, it was assumed that the response rate was respectively 10% under the null hypothesis, and that the response rate was 25% under the alternative hypothesis, in SCLC cohort.

For SCLC cohort, if at least 2 responses (confirmed CR (complete response)+PR (partial response)) were observed on the first 25 treated patients, accrual could continue to the full sample size of 51 patients treated.

The study cut-off date was when the last patient on treatment in the cohort had two tumor assessments or end of treatment tumor assessment, whichever occurred first in order to assess antitumor activity.

### Study Group

For SCLC, the recruitment was restricted to diseases for which CEACAM5 expression in the most recent FFPE archival tumor tissue sample is ≥2+ in intensity in ≥1% of the tumor cell population, as demonstrated prospectively by local or central IHC evaluation.

The total study group was made of 26 patients. The SCLC cohort included patients with CEACAM5 expression of ≥2+ in intensity involving ≥1% of the tumor cell population.

Patients had a median age of 67.0 years, with a range of 49 to 81 years, with 57.7% aged 65 years and older. There were more males than females overall, (65.4% male and 34.6% female), most of which were white/caucasian (73.1%). Most (80.8%) of the patients entered with an ECOG score of 1, 15.4% entered with an ECOG score of 0, and 1 patient (3.8%) had an ECOG score of 2.

Inclusion criteria were: Locally advanced or metastatic solid malignant tumor disease for which, in the judgment of the Investigator, no standard alternative therapy was available, and meeting the following inclusion criteria:
Patients with SCLC with CEACAM5 expression in the most recent FFPE archival tumor tissue sample, is ≥2+ in intensity involving ≥1% of the tumor cell population, as demonstrated prospectively by local or central IHC evaluation;
At least 6×5 μm slides plus an additional number of slides that could be 3×10 m (best) or 6×5 μm or equivalent to keep the same total amount of material needed, from formalin-fixed paraffin-embedded (FFPE) archival tissue should be available for local testing at the site and/or shipment to the Sponsor, or laboratory designated by the Sponsor, evaluation of tumor CEACAM5 expression (retrospectively in the Escalation Phase and prospectively in the Expansion Phase) and exploration of other predictive biomarkers of response. If less material is available, patient could still be eligible after discussion with the Sponsor who will assess and confirm that there is sufficient relevant material for key evaluations.

Inclusion is enriched for (although not restricted to) tumors expressing or likely to be expressing CEACAM5 which includes:
Malignant diseases with high prevalence of CEACAM5 expression ie, non-sqNSCLC of the adenocarcinoma or large cell subtype, or,•
Circulating Carcinoembryonic Antigen (CEA) levels >5 ng/mL, as demonstrated by local testing.

Recruitment was restricted to diseases for which CEACAM5 expression in the most recent FFPE archival tumor tissue sample is ≥2+ in intensity in ≥1% of the tumor cell population.

Of the 26 patients in the small cell lung cancer expansion phase, all experienced metastatic disease, and all had measurable disease. The most common organs involved included lung (69.2%), lymph node (57.7%), liver (42.3%), brain (34.6%), pleura (26.9%), adrenal gland (23.1%), and bone (19.2%). Slightly more than half (53.8%) of patients were sensitive and 46.2% were refractory. CEACAM5 expression at intensity 2+/3+ on archival sample was ≥50% in 46.2% patients (30.8% had 50-80% expression and 15.4% had ≥80% expression). Median circulating CEA level was 16.00 μg/L with a range of 0.8 to 5016.3 g/L. Five patients (21.7%) had circulating CEA levels ≥100 μg/L.

The level of CEACAM5 expression was documented essentially retrospectively and centrally on both archival and fresh (baseline sample) tumor tissues.

Confirmation of CEACAM5 tumor expression was done retrospectively in a central laboratory on fresh tumor tissue collected at baseline. If sufficient archived tumor material was available, a central evaluation was also done retrospectively to gain knowledge on the variability in evaluation of the expression. The results of the retrospective analyses had no impact on patient's treatment. It was for the better interpretation of the overall response, and as the baseline for comparison with CEACAM5 expression upon progression (exploration for loss of CEACAM5 as mechanism of acquired resistance).

The duration of the study for an individual patient included a period for inclusion of up to 4 weeks (baseline period), a treatment period of at least 1 cycle (2 weeks), an end-of-treatment (EOT) visit around 30 days following the last investigational medical product (IMP) administration, and at least one follow-up visit (around 30 days after EOT visit) for immunogenicity evaluation.

### Pre-Treatment

All 26 patients in the small cell lung cancer expansion phase received chemotherapy regimen prior to the study. The median number of prior regimens was 2 (range of 1 to 6) with 42.3% of patients heavily pretreated and having ≥3 prior regimens. Most (69.2%) patients had no antitubulin agent exposure prior to the study, of the 30.8% of patients who did use anti-tubulin agents, 87.5% had prior taxane exposure, and 25.0% had prior vinca-alkaloid exposure. Most patients with anti-tubulin agent exposure had 1 prior regimen (7/8 patients, 87.5%). Most (76.9%) patients had no prior anti PD1/PDL1 agent exposure.

### Investigational Product

Tusamitamab ravtansine (SAR408701) was administered, based on actual body surface area, intravenously as Q2W cycle with or without loading dose at Cycle 1., depending on the cohorts that patient was enrolled.

The drug was administered every 2 weeks at 100 mg/m².

The patient's body surface area (BSA) was calculated using their height and actual body weight. For patients with a BSA >2.2 m², the dose was calculated on the basis of 2.2 m² BSA.

Pre-medication with Histamine H1 antagonist (diphenylhydramine 50 mg PO or equivalent [e.g., dexchlorpheniramine] given approximately 1 hour before tusamitamab ravtansine administration) was required for all patients.

The tusamitamab ravtansine was supplied as a 25 mL extractable volume of concentrate for solution for infusion of 125 mg (5 mg/mL) contained in a 30 mL type I glass vial.

Using an infusion controlled pump, tusamitamab ravtansine was administered by IV infusion at a rate of 2.5 mg/min for the first 30 minutes and then increased to 5 mg/min in the absence of hypersensitivity reactions.

Tusamitamab ravtansine was administered on Day 1 and repeated every 14 days; this period of 14 days constituted one treatment cycle (1 cycle). Patients were able to continue treatment until disease progression, unacceptable toxicity, or willingness to stop.

No bacteriostatic agent was present in the product; therefore, adherence to aseptic technique was required. Prior to dosing, each patient's dose needs to be individually prepared by the study pharmacist starting from pre-filled bags of diluent (0.9% sodium chloride).

Once the solution is prepared, the dose was administered to the patient within 7.5 hours from the bag preparation to the end of the dose infusion.

Two types of administration were used:
Infusion by syringe driver for low doses (up to 30 mg/m2).
Infusion by pump for other doses.

An IV tubing administration set with a 0.2 micron filter unit attached to it was used for infusion. The study drug was not administered with any other IV fluids. However, infusion tubing was optionally primed with normal saline or tusamitamab ravtansine. For infused volumes <25 mL, a flush and destruction of 25 mL of tusamitamab ravtansine needed to be ensured before infusion of the dose. At the end of the infusion by pump, the IV line was flushed with normal saline as needed to ensure delivery of the full dose. At the end of the infusion by syringe driver, the remaining quantity of tusamitamab ravtansine in the syringe was destroyed.

### Safety

Of the 26 patients in the small cell lung cancer expansion phase, all experienced at least one TEAE (treatment-emergent adverse event), with 61.5% experiencing a Grade 3 or higher TEAE. Six patients (23.1%) experienced a Grade 5 TEAE (context of disease progression for all 6 patients: 5 patients experienced disease progression, and 1 patient had respiratory failure). Fifteen (57.7%) patients experienced a serious TEAE. A majority of patients (80.8%) experienced TEAEs related to the study drug, 19.2% of patients experienced related TEAEs Grade 3 or higher (none of which were Grade 5). One patient (3.8%) experienced a serious related TEAE (Grade ≥3 AST increased) Six patients (23.1%) experienced a TEAE leading to dose modification, with no patient experiencing a TEAE leading to permanent treatment discontinuation.

For the 26 patients in the small cell lung cancer expansion phase, TEAEs of any Grade and regardless of relationship with study treatment were most frequently reported in the SOCs of General disorders and administration site conditions (61.5%), Respiratory, thoracic and mediastinal disorders (53.8%), Nervous system disorders (50.0%), Gastrointestinal disorders (46.2%), Metabolism and nutrition disorders (42.3%) and Investigations (42.3%).

The most common TEAEs of any Grade, regardless of relationship with study treatment, and reported with a frequency over 10% were: Asthenia (34.6%), Dyspnea (30.8%), Decreased appetite (26.9%), Disease progression (23.1%), AST increase, constipation, keratitis, nausea (19.2%), Cough (15.4%), Dizziness, hyponatraemia, oedema peripheral, peripheral sensory neuropathy, pruritis, and rash (11.5%).

No patients in small cell lung cancer cohort experienced a TEAE leading to permanent treatment discontinuation.

### Efficacy Measurement

All patients treated in the Expansion Phase had to have at least one measurable lesion for inclusion as efficacy was primary objective. Tumor assessments were made at least every 4 cycles. Decision to pursue treatment was based on the response evaluation made by the Investigator, however, measures of lesions were collected in the e-CRF for a determination of response by Sponsor. A partial or complete response must have been confirmed on a second examination done at least 4 weeks apart, in order to be documented as a confirmed response to therapy.

The primary efficacy endpoint in expansion phase was overall objective response, which was defined as a confirmed CR (complete response) or a PR (partial response), evaluated every 4 cycles using RECIST v1.1. Secondary efficacy variables included duration of response and TTP for expansion phase.

### Response-Evaluable Population

The response-evaluable population was defined as all treated patients who had measurable disease at study entry and at least 1 post-baseline evaluable tumor assessment. Response evaluable patients with Small cell lung also had to have had CEACAM5-expressing malignancies documented locally on archival tumor tissue and meeting criteria.

### Tumor Response Evaluation

To assess objective response or future progression, it was necessary to estimate the overall tumor burden at baseline and use this as a comparator for subsequent measurements. Only patients with measurable disease at baseline were included in protocols where objective tumor response was the primary endpoint. Measurable disease was defined by the presence of at least one measurable lesion. In studies where the primary endpoint was tumor progression (either time to progression or proportion with progression at a fixed date), the protocol specified if entry is restricted to those with measurable disease or whether patients having non-measurable disease only are also eligible.

### Response Criteria

The response criteria that was applied is provided in Table 7 and Table 8 below.

TABLE 7
Evaluation of target lesions

| | |
|---|---|
| Complete | Disappearance of all target lesions. Any pathological |
| Response | lymph nodes (whether target or non-target) must |
| (CR) | have reduction in short axis to <10 mm. |
| Partial | At least a 30% decrease in the sum of diameters of |
| Response | target lesions, taking as reference the baseline |
| (PR) | sum diameters. |
| Progressive | At least a 20% increase in the sum of diameters of |
| Disease (PD) | target lesions, taking as reference the smallest |
| | sum on study (this includes the baseline sum if that |
| | is the smallest on study). In addition to the relative |
| | increase of 20%, the sum must also demonstrate an |
| | absolute increase of at least 5 mm. (Note: the |
| | appearance of one or more new lesions is also |
| | considered progression). |
| Stable | Neither sufficient shrinkage to qualify for PR nor |
| Disease | sufficient increase to qualify for PD, taking as |
| (SD) | reference the smallest sum diameters while on study. |

TABLE 8
Evaluation of non-target lesions

| | |
|---|---|
| | Disappearance of all non-target lesions and |
| | normalization of tumor marker level. All |
| | lymph nodes must be non-pathological in size |
| | (<10 mm short axis). |
| Incomplete | Persistence of one or more non-target lesion(s) |
| | and/or maintenance of tumour marker level |
| Response/Stable | Above the normal limits. |
| Disease (SD) | |
| Progressive | Unequivocal progression (see comments below) of |
| Disease (PD) | existing non-target lesions. (Note: the appearance |
| | of one or more new lesions is also considered |
| | progression). |

Though a clear progression of “non-target” lesions only is exceptional, in such circumstances, the opinion of the treating physician should prevail and the progression status should be confirmed later on by the review panel (or study chair).

### Evaluation of Best Overall Response

Of the 26 patients in the small cell lung cancer expansion phase treated at the recommended dose from the dose escalation phase of 100 mg/m2 Q2W (every two weeks), 1 patient (3.8%) achieved partial response at Cycle 12. The Overall Response Rate (ORR) was 3.8%. Nine patients (34.6%) had as best overall response of SD; of these, 1 patient had no confirmed PR (−43.75% reduction).

Table 9 below provides a summary of the overall response status calculation at each time point for patients who have measurable disease at baseline.

TABLE 9
(Best overall Response and Overall Response Rate
all treated population - expansion phase SCLC)

Tusamitamab
ravtansine
100 mg/m²
QS2W (n = 26)

Best overall response [n(%)]

| Complete response (CR) | 0 | |
|---|---|---|
| Partial response (PR)^{a} | 1 | (3.8%) |
| Stable disease | 9 | (34.6%) |
| Progressive disease (PD) | 15 | (57.5%) |
| Not evaluable^{b} | 1 | (3.8%) |
| Overall response rate (confirmed CR and PR) (%) | 1 | (3.8%) |

| | |
|---|---|
| 95% CI^{c} | (0.68%-18.89%) |
| p-Value^{d} | 0.7487 |

^{a}Confirmation of response is required (ie a second examination done at least 4 weekend apart (ie ≥28 days) in order to be documented as a confirmed response)
^{b}Including patients with no post-baseline evaluation due to early death or early progression based on symptomatic deterioration
^{c}Estimated by Wilson score interval
^{d}A binomial test against the null hypothesis of response rate of 10% is performed using a one-sided 0.05 alpha level

TABLE 10
(Summary of CEACAM5 prevalence at pre-screening
and responses following tusamitamab ravtansine
treatment - TED13751 SCLC cohort)

| | Number of prescreened | Number of treated | Observed |
|---|---|---|---|
| Cohort | patients | patients | responses |
| SCLC | N = 279 | 26 | CR: 0, PR: 1, |
| | ≥1% (N = 76): 27.2% | | SD: 9 |
| | ≥25% (N = 41): 14.7% | | |
| | ≥50% (N = 31): 11.1% | | |

If a complete regression (CR) was truly met at first time point, then any disease seen at a subsequent time point, even disease meeting partial regression (PR) criteria relative to baseline, made the disease progression disease (PD) at that point (since disease must have reappeared after CR). Best response would depend on whether minimum duration for stable disease (SD) was met. However, sometimes ‘CR’ could be claimed when subsequent scans suggested small lesions were likely still present and in fact the patient had PR, not CR at the first time point. Under these circumstances, the original CR should have been changed to PR and the best response is PR.

The best overall response was determined once all the data for the patient was known.

Best response determination in trials where confirmation of complete or partial response was not required: Best response in these trials was defined as the best response across all time points (for example, a patient who has SD at first assessment, PR at second assessment, and PD on last assessment has a best overall response of PR). When SD was believed to be best response, it also needed to meet the protocol specified minimum time from baseline. If the minimum time was not met when SD was otherwise the best time point response, the patient's best response depended on the subsequent assessments. For example, a patient who had SD at first assessment, PD at second and did not meet minimum duration for SD, would have a best response of PD. The same patient lost to follow-up after the first SD assessment would have been considered non-evaluable.

Best response determination in trials were confirmation of complete or partial response was required: Complete or partial responses could be claimed only if the criteria for each were met at a subsequent time point as specified in the protocol (generally 4 weeks later). In this circumstance, the best overall response was interpreted as in Table 7.

### Duration of Response

For the 26 patients in the small cell lung cancer expansion phase, the median duration of study treatment was 8.50 weeks, with a range of 2.0 to 52.6 weeks. The median number of cycles administered per patient was 4 cycles, with a range of 1 to 26 cycles. For the only patient in the small cell lung cancer cohort who responded, the duration of response was 6.2 months.

### Time of Progression

Median time to tumor progression was 1.9 months (95% CI of 1.68-2.73) with 1 patient being censored.

### CEACAM5—Immunohistochemistry (IHC) Scoring Method

CEACAM5 stained immunohistochemical slides were assessed by a pathologist by light microscopy.

CEACAM5 positivity was determined by the percentage of viable tumor cells expressing CEACAM5 positive membrane staining.

Tumor cells were CEACAM5 positive if they exhibited either partial or complete circumferential plasma membrane staining at 2+ and 3+ intensities. They were considered as negative if they exhibited staining at 1+ intensity (weak staining) or no staining (intensity 0).

All tumor cells observed on the section were evaluated for CEACAM5.

A minimum of 100 viable tumor cells needed to be present on the section to determine the percentage of CEACAM5 positive cells.

Scoring captures the percentage of tumor cells stained at each intensity measured as indicated below:

$$\% CEACAM 5 positive = 100 \times \# of tumor cells expressing CEACAM 5 membrane staining at \geq 2 + intensity / Total \# of viable tumor cells present in the section .$$

On the archival sample, 57.1% of patients expressed CEACAM5 with intensity ≥2+ in less than 50% of the tumoral cells, whereas 42.9% of patients expressed CEACAM5 with intensity ≥2+ in at least 50% of the tumoral cells as per central assessment.

### Results

### Expression CEACAM5

archived tumor samples, percentage of positive cells of For CEACAM5 at intensity ≥2+ comparing whole membrane results to polarized membrane results are presented in Table 11. The main pattern of CEACAM5 expression in SCLC was whole membrane.

TABLE 11
(Summary table of percentage of positive cells of CEACAM5
at intensity ≥2+ comparing Whole membrane results
to Polarized membrane results)

Tusamitamab ravtansine 100
mg/m² Q2W (N = 26)

| | |
|---|---|
| Whole | Polarized |
| membrane | membrane |
| (N = 26) | (N = 26) |

Percentage of positive cells of CEACAM5 at intensity ≥2+

| | | |
|---|---|---|
| Number | 26 | 26 |
| Mean (SD) | 39.88 (34.12) | 0.69 (2.46) |
| Median | 40.00 | 0.00 |
| Min:Max | 0.0:100.0 | 0.0:10.0 |

### Tumor Shrinkage

Best relative tumor shrinkage by class of percentage of positive cells at intensity ≥2+, archival biopsy, overall membrane is presented in Table 12. No association was observed between the best relative tumor shrinkage and the expression of CEACAM5 at intensity ≥2+ in the archive sample (adjusted p-value, 0.2877), but there was an apparent trend for better tumor shrinkage with higher CEACAM5 expression.

TABLE 12
(Best relative tumor shrinkage by class of percentage of positive cells
at intensity ≥2+, archival biopsy, overall membrane)

| Tusamitamab ravtansine 100 mg/m² Q2W (N = 26) | | | | |
|---|---|---|---|---|
| Percentage of positive cells of CEACAM5 at intensity ≥2+ | | | | |
| <50 | [50-80[(N = | ≥80 | p- | Adjusted |
| (N = 14) | 8) | (N = 4) | Value* | p-value** |

Best relative tumor shrinkage

| | | | | | |
|---|---|---|---|---|---|
| Number | 14 | 7 | 4 | 0.0137 | 0.2877 |
| Mean (SD) | 23.6 | −6.2 | −12.1 | | |
| | (28.1) | (32.9) | (21.8) | | |
| Median | 17.4 | −16.3 | −4.8 | | |
| Min:Max | −10:98 | −38:58 | −44:5 | | |

*A Kruskall-Wallis test was performed
**the adjusted p-values were calculated with the application of Benjamini-Hochberg multiple correction procedure to control false discovery rate.

A moderate association was observed between the best relative tumor shrinkage and the expression of CEACAM5 at intensity ≥2+ in the archive sample (−0.53 adjusted p-value of 0.2877 non statistically significant), but it seemed to be a trend for better tumor shrinkage with higher CEACAM5 expression. NB: CEACAM5 prevalence at ≥50% 2+/3+ threshold was 11.1%.

In the SCLC cohort, out of the 26 treated patients, 1 confirmed partial response was reported. The overall response rate was 3.8% [95% CI: 0.68-18.89%]). Nine patients reported a Best Overall Response (BOR) of SD. Duration of response for the patient with PR was 6.2 months. Median time to progression was 1.9 months.

### CONCLUSION

In conclusion, these data demonstrate that proof of concept was achieved in a subset of neuroendocrine small cell lung cancers treated with tusamitamab ravtansine. In particular, these data support the conclusion that tusamitamab ravtansine is effective in treating neuroendocrine SCLC. Moreover, these data support the conclusion that tusamitamab ravtansine is particularly effective in treating high CEACAM5 expressing neuroendocrine SCLC.

1. An immunoconjugate comprising an antibody or an antigen-binding fragment thereof, for use in treating a cancer selected from neuroendocrine cancers expressing hCEACAM5, in a subject in need thereof, wherein said antibody or antigen-binding fragment thereof specifically binds hCEACAM5, and wherein said antibody or antigen-binding fragment thereof is conjugated or linked to at least one growth inhibitory agent, wherein said growth inhibitory agent is not a topoisomerase I inhibitor.

2. The immunoconjugate for use according to claim 1, wherein said antibody or antigen-binding fragment thereof comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

3. An antibody, or an antigen-binding fragment thereof, or an immunoconjugate comprising said antibody or said antigen-binding fragment thereof, for use in treating a cancer selected from neuroendocrine cancers expressing hCEACAM5, in a subject in need thereof, wherein said antibody or antigen-binding fragment thereof specifically binds hCEACAM5, wherein said antibody or antigen-binding fragment thereof comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT),
wherein said neuroendocrine cancer is not a neuroendocrine small cell lung carcinoma (SCLC).

4. An antibody, or an antigen-binding fragment thereof, or an immunoconjugate comprising said antibody or said antigen-binding fragment, for use in treating a cancer selected from neuroendocrine cancers expressing hCEACAM5 in a subject in need thereof, wherein the antibody specifically binds hCEACAM5 and wherein the antibody, or the antigen-binding fragment thereof, comprises a VH and a VL domain, wherein the VH domain comprises the three complementarity-determining regions (CDRs) HCDR1, HCDR2 and HCDR3 and wherein the VL domain comprises the three CDRs: LCDR1, LCDR2 and LCDR3, wherein
the HCDR1 comprises the amino acid sequence of SEQ ID NO: 3 (GFVFSSYD); the HCDR2 comprises the amino acid sequence of SEQ ID NO: 4 (ISSGGGIT); the HCDR3 comprises the amino acid sequence of SEQ ID NO: 5 (AAHYFGSSGPFAY); and
the LCDR1 comprises the amino acid sequence of SEQ ID NO: 6 (ENIFSY); the LCDR2 comprises the amino acid sequence of NTR; and the LCDR3 comprises the amino acid sequence of SEQ ID NO: 7 (QHHYGTPFT).

5. The antibody or immunoconjugate for use according to any one of claims 1-4, wherein the cancer is selected from neuroendocrine tumors (NET), neuroendocrine carcinomas (NEC), Mixed Neuroendocrine-Non-Neuroendocrine Neoplasma (MiNEN), Pheochromocytoma and Medullary thyroid carcinoma (MTC).

6. The antibody or immunoconjugate for use according to any one of claims 1, 2 and 4, wherein the cancer is selected from neuroendocrine cancers of:
the gastrointestinal and pancreatobiliary tract, such as neuroendocrine cancers of the digestive tract in particular the oesophagus, the stomach, the pancreas, the liver, the small intestine and large intestine or the anal region, or such as neuroendocrine cancers of the urinary tract, in particular the bladder;
the upper aerodigestive tract and salivary glands, such as neuroendocrine cancers of the lung, in particular a small cell lung cancer, and such as neuroendocrine cancers of the thymus;
the thyroid, such as medullary thyroid carcinoma (MTC);
the adrenal gland, such as pheochromocytoma;
the skin, such as Merkel cell carcinoma (MCC);
the female reproductive organs, such as of the endometrium, the uterine cervix or the ovary;
the male reproductive organs, such as of the prostate or the testis, in particular the testis, and
the head and neck.

7. The antibody or immunoconjugate for use according to any one of claims 1, 2 and 4, wherein the cancer is selected from neuroendocrine cancers of the oesophagus, the stomach, the small intestine and the large intestine, the anal region, the pancreas, the bladder, the lung, the female reproductive organs, the male reproductive organs, the thyroid and the head and neck.

8. The antibody or immunoconjugate for use according to any one of claims 1, 2 and 4, wherein the cancer is a neuroendocrine cancer of the lung, in particular a neuroendocrine small cell lung cancer, or a neuroendocrine cancer of the male reproductive organs, in particular a neuroendocrine prostate cancer.

9. The antibody or immunoconjugate for use according to any one of claims 1, 2 and 4-8, wherein the cancer is a neuroendocrine small cell lung cancer.

10. The antibody or immunoconjugate for use according to any one of claims 1, 2 and 4-8, wherein the cancer is neuroendocrine prostate cancer.

11. The antibody or immunoconjugate for use according to any one of the preceding claims, wherein the subject is a moderate or high carcinoembryonic antigen-related cell adhesion molecule expresser.

12. The antibody or immunoconjugate for use according to claim 11, wherein the subject is a moderate CEACAM5 expresser, that is a subject who has a CEACAM5 expression consisting of 2+ or 3+ intensity, measured by hCEACAM5 immunohistochemistry, in at least 1% of the tumor cell population or consisting of 1+ intensity in at least 50% of the tumor cell population.

13. The antibody or immunoconjugate for use according to claim 11, wherein the subject is a high CEACAM5 expresser, that is a subject who has a CEACAM5 expression consisting of 2+ or 3+ intensity, measured by hCEACAM5 immunohistochemistry, in over 50% of the tumor cell population.

14. The antibody or immunoconjugate for use according to any one of claims 2 to 13, wherein the VH domain comprises SEQ ID NO: 1:
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSS.

15. The antibody or immunoconjugate for use according to any one of the preceding claims, comprising a heavy chain comprising SEQ ID NO: 8:
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

16. The antibody or immunoconjugate for use according to any one of claims 2 to 15, wherein the VL domain comprises SEQ ID NO: 2:
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIK.

17. The antibody or immunoconjugate for use according to any one of claims 2 to 16, comprising a light chain comprising SEQ ID NO: 9:
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

18. The immunoconjugate comprising the antibody, for use according to any one of claims 3 to 17, wherein the antibody is conjugated or linked to at least one growth inhibitory agent, in particular to at least one growth inhibitor that is a cytotoxic agent.

19. The immunoconjugate for use according to claim 18, wherein said growth inhibitory agent is selected from the group consisting of chemotherapeutic agents, enzymes, antibiotics, and toxins such as small molecule toxins or enzymatically active toxins, taxoids, vincas, taxanes, maytansinoid or maytansinoid analogs, tomaymycin or pyrrolobenzodiazepine derivatives, cryptophycin derivatives, leptomycin derivatives, auristatin or dolastatin analogs, prodrugs, topoisomerase inhibitors, DNA alkylating agents, anti-tubulin agents, and CC-1065 or CC-1065 analogs.

20. The immunoconjugate for use according to claim 18, wherein said growth inhibitor is not a topoisomerase I inhibitor.

21. The immunoconjugate for use according to any one of claims 1-2 and 5-20, wherein said growth inhibitory agent is (N²′-deacetyl-N²′-(3-mercapto-1-oxopropyl)-maytansine) (DM1) or N²′-deacetyl-N²′-(4-methyl-4-mercapto-1-oxopentyl)-maytansine (DM4).

22. The immunoconjugate for use according to any one of claims 1-2 and 5-21, wherein the antibody is covalently attached via a cleavable or non-cleavable linker to the at least one growth inhibitory agent, in particular via a linker selected from the group consisting of N-succinimidyl pyridyldithiobutyrate (SPDB), 4-(Pyridin-2-yldisulfanyl)-2-sulfo-butyric acid (sulfo-SPDB), and succinimidyl (N-maleimidomethyl) cyclohexane-1-carboxylate (SMCC).

23. The immunoconjugate for use according to any one of claims 1-22, comprising a hCEACAM5-antibody, which comprises a heavy chain consisting of SEQ ID NO: 8 and a light chain consisting of SEQ ID NO: 9, and which is covalently linked to N²′-deacetyl-N²′(4-methyl-4-mercapto-1-oxopentyl)-maytansine (DM4) via N-succinimidyl pyridyldithiobutyrate (SPDB).

24. The immunoconjugate for use according to any one of claims 1-23, wherein the immunoconjugate is Tusamitamab ravtansine.

25. The antibody or immunoconjugate for use according to any one of claims 1-24, wherein the antibody or immunoconjugate is administered at a dose level of 5, 10, 20, 30, 40, 60, 80, 100, 120, 150, 180, or 210 mg/m² based on the body surface area of the subject.

26. The antibody or immunoconjugate for use according to any one of claims 1-25, wherein the antibody or immunoconjugate is administered every 2 weeks, or every 3 weeks.

27. The antibody or immunoconjugate for use according to any of claims 1-26, wherein the antibody or immunoconjugate is administered at a dose level of 100 mg/m² based on the body surface area of the subject every 2 weeks.

28. The antibody or immunoconjugate for use according to any one of claims 1-27, wherein the antibody or immunoconjugate is administered at a dose of from about 100 mg/m² to about 200 mg/m² as a loading dose, in particular of about 135 mg/m², about 150 mg/m² or about 170 mg/m², as a loading dose.
